(19) United States
(12) Patent Application Publication
(10) Pub. No.: US 2011/0040192 A1
(43) Pub. Date: Feb. 17, 2011

(54) METHOD AND A SYSTEM FOR IMAGING AND ANALYSIS FOR MOLE EVOLUTION TRACKING

(76) Inventors: Sara Brenner, Tel Aviv (IL); Itzhak Pomerantz, Kefar Sava (IL); Yariv Oz, Petach Tikva (IL); Einat Kidron, Tel Mond (IL); Victor Taubkin, Petach Tikva (IL)

Correspondence Address: The Law Office of Michael E. Kondoudis, 888 16th Street, N.W., Suite 800, Washington, DC, 20006, US

(21) Appl. No.: 12/785,225

(22) Filed: May 21, 2010

Related U.S. Application Data
(60) Provisional application No. 61/180,135, filed on May 21, 2009.

(51) Int. Cl. A61B 6/00; G06K 9/00; H04N 7/18

(52) U.S. Cl. 600/476; 382/128; 348/77; 348/E07.085

(57) ABSTRACT

A mole monitoring system including: (a) a calibration fixture including an illuminator that is operational to illuminate a skin area that includes a mole; (b) a digital camera operational to acquire at least one image of a body area that is included in the illuminated skin area and which includes the mole and its environment; and (c) a processor operational to analyze the at least one image of the mole and to determine values for parameters of the mole.

### BACKGROUND OF THE INVENTION

Melanoma is a well known type of skin cancer. Moles on the skin are known in the art of dermatology as a reliable Precursors of Melanoma. Melanoma can arise in a congenital mole in up to 85% or denovo. Certain populations (such as light skin, blue eyes, red hair) are considered to have a higher-risk of developing melanoma, and therefore the routine monitoring of moles is a well known practice in dermatology. The prior art practice of monitoring moles as an early warning on melanoma is widely described in the medical literature and can be found, for example, in the publications of the Cancer Research Institute, located in 681 Fifth Avenue, New York, N.Y., USA.

Academic literature about the relationship of mole appearance and melanoma can be found, inter alia, in the following publications:

Argenziano G, Soyer H P, Chimenti S, et al. 2003. Dermoscopy of pigmented skin lesions: results of a consensus meeting via the Internet. J Am Acad Dermatol 48:679-693.

Elbaum M. 2002. Computer-aided melanoma diagnosis. Dermatol Clin 20:735-747, x-xi.

Rosado B, Menzies S, Harbauer A, et al. 2003. Accuracy of computer diagnosis of melanoma: a quantitative meta-analysis. Arch Dermatol 139:361-367.

Several technologies, patents and products are offered in the prior art for the purpose of detecting suspicious changes in the appearance of moles. Some of these technologies are described in the U.S. Pat. No. 6,427,022 “Image comparator system and method for detecting changes in skin lesions” to Craine and in the U.S. Pat. No. 4,938,948 “Method for imaging breast tumors using labeled monoclonal anti-human breast cancer antibodies” to Ring and others.

The prior art technologies are seeking to detect symptoms of Melanoma and are teaching complex and expensive systems that require professional operators. They are typically intended for the clinic and the hospital.

However, it is well accepted among dermatologists, that the moles of people in high risk groups should be inspected much more frequently than the typical frequency of visiting a doctor. Unfortunately, this requirement is not practical and the compromise may cause belated detection of cancer and risk the patient.

Some systems that are available today are intended for physician office use, and are making detailed digital photographs of the moles, enabling the physician to inspect the mole remotely and seek visual evidence of deformations of moles. Such system, called “MoleMate” is available from Astron Clinica Limited, The Mount, Toft, Cambridge, UK. However, this product does not alert the patient at home that his moles need to visit the physician—and leave all the load of screening on the physician.

It would be very desirable to have a method and a system that enable home monitoring of mole evolution and alert a patient that his mole is deforming and that he should accelerate his visit to the physician.

### SUMMERY OF THE INVENTION

An imaging system for imaging a body area, the system including: (a) an air blower for moving hair of the body area between different locations by blowing air toward the hair; wherein the hair covers different segments of the body area while being positioned at different locations; (b) a camera, configured to acquire multiple images of the body area while the hair is positioned in different locations due to the blowing of air; and (c) a processor, configured to: (i) receive the multiple images; (ii) extract information of pixels from the multiple images based on an amount of hair information included in the pixels; and (iii) generate a synthetic image of the body area, in which different pixels are extracted from different images of the multiple images.

A mole monitoring system including: (a) a calibration fixture including an illuminator that is operational to illuminate a skin area that includes a mole; (b) a digital camera operational to acquire at least one image of a body area that is included in the illuminated skin area and which includes the mole and its environment; and (c) a processor operational to analyze the at least one image of the mole and to determine values for parameters of the mole.

A computer program product including a non-transient computer readable medium that stores instructions for: (a) acquiring from a camera visual information in a monitored field of view; (b) automatically detecting a mole in the field of view; (c) automatically analyzing the visual information for measuring multiple independent parameters of the mole; (d) automatically identifying the mole, by its measured parameters, among a database including information of multiple moles; and (e) automatically comparing the measured parameters to previously measured parameters of the mole, for detecting trends of change in at least one of the measured parameters along a sequence of at least three measurements of the mole at different times.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates imaging a system for imaging a body area, according to an embodiment of the invention;

FIG. 2 illustrates a method for imaging a body area, according to an embodiment of the invention;

FIGS. 3A through 3D exemplify the parameter of mole asymmetry;

FIGS. 3E through 3G exemplify the parameter of border roughness;

FIGS. 3H and 3I exemplify the parameter of variation in color;

FIG. 4 exemplify the parameter of mole diameter;

FIG. 5 exemplify the parameter of peripheral mole redness;

FIG. 6 exemplify the parameter of mole topography;

FIG. 7 is a block diagram of a system, according to an embodiment of the invention;

FIG. 8 illustrates a method, according to an embodiment of the invention;

FIG. 9 illustrates a flowchart of a capture process, according to an embodiment of the invention;

FIG. 10 illustrates a flowchart of an analysis process, according to an embodiment of the invention;

FIG. 11 illustrates a flowchart of a process of determining asymmetry of a mole, according to an embodiment of the invention;

FIG. 12 illustrates a flowchart of a process of determining a border irregularity of a mole, according to an embodiment of the invention;

FIG. 13 illustrates a field of view of a camera including calibration patches, according to an embodiment of the invention;

FIG. 14 illustrates a simplified flowchart of a process of determining a color of a mole, according to an embodiment of the invention;

FIG. 15 illustrates a flowchart of a process of determining a diameter of a mole, according to an embodiment of the invention;

FIG. 16 illustrates a simplified flowchart of a process of determining a topography of a mole, according to an embodiment of the invention;

FIG. 17 illustrates a flowchart of a process of identifying a mole among the moles of a patient, according to an embodiment of the invention;

FIG. 18 illustrates a flowchart of a process of detection of an evolution in parameters of a mole, according to an embodiment of the invention;

FIG. 19 illustrates light switching measurement of topography, according to an embodiment of the invention;

FIG. 20 illustrates a system, according to an embodiment of the invention;

FIG. 21 illustrates a vertical cross section of a support fixture, according to an embodiment of the invention;

FIG. 22 illustrates utilization of a general purpose web camera connected to a computer, according to an embodiment of the invention;

FIG. 23 illustrates a process that may be implemented by a computer program product, according to an embodiment of the invention;

FIG. 24 illustrates a portable mole evolution monitoring system, according to an embodiment of the invention; and

FIG. 25 illustrates a mole monitoring system, according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

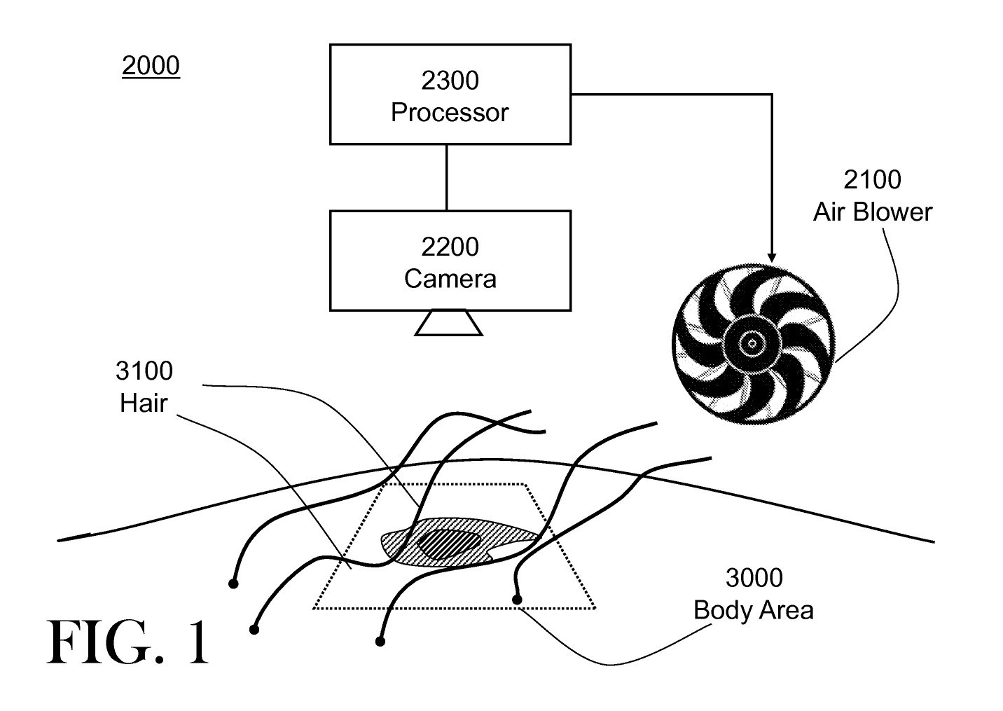

FIG. 1 illustrates imaging system 2000 for imaging body area 3000, according to an embodiment of the invention. It is noted that conveniently the body area is a skin area of a human body. It is noted that body area 3000 may contain a mole.

System 2000 includes air blower 2100 for moving hair (i.e. one or more hairs) 3100 of body area 3000 between different locations by blowing air toward hair 3100; wherein hair 3100 covers different segments of body area 3000 while being positioned at different locations. It is noted that hair 3100 of body area 3000 is either hair originating in body area 3000, and/or hair of the body which is at least temporarily located (and/or placed) on body area 3000 and may interrupt with clear imaging thereof (e.g. by blocking a line of sight between camera 2200 and the skin of body area 3000 that lies under the hair)

It is noted that while some of the following descriptions related to situations in which hair 3100 includes one or few hairs (e.g. hair sprouting from a mole), various embodiments of system 2000 are adapted to operate when hair 3100 include numerous hairs (e.g. hair locks).

It is noted that, according to some embodiments of the invention, system 2000 may use an external air blower (or equivalent source of wind or of compressed air) instead of integral air blower 2100. according to various embodiments of the invention, Air blower 2100 may be focused (e.g. similar to a hair drier) and may be unfocused (e.g. similar to a fan). Air blower 2100 may be controlled manually or automatically.

System 2000 further includes camera 2200 that is configured to acquire multiple images of body area 3000 (e.g. by capturing a short video sequence) while hair 3100 (i.e. all of, or at least few hairs of which) is positioned in different locations due to the blowing of air. The timing of the image acquisitioned of each image may be programmed in advanced (e.g. with respect to the timing of the first image acquired), may be determined automatically (e.g. by analysis of previously acquired images), and may be determined manually. By way of example, 10 frames (snapshots) that are taken at intervals of 0.1 seconds apart may be sufficient, according to an embodiment of the invention, for successful generation of synthetic image, as disclosed below.

It is noted that all of the images may conveniently be acquired when camera 2200 is still, and/or is fixed in respect to body area 3100 (e.g. camera 2200 may be lean against body area 3100 or a near by body area). Likewise, the lighting for all of the multiple images may conveniently be substantially identical (even though compensation may be implemented in a later stage).

System 2000 further includes processor 2300 that is configured to receive the multiple images and to process them. It is noted that according to various embodiments of the invention, processor 2300 may be implemented as a group of two or more processors that can communicate among which. For example, some of the activities disclosed in relation to processor 2300 may be implemented by a processor of camera 2200.

Furthermore, it is noted that according to an embodiment of the invention, processor 2300 and camera 2200 may be part of a single unit (e.g. enclosed within the same casing, e.g. both part of a smart phone), and that according to other embodiments of the invention processor 2300 may be included in a different unit, and receive information from camera 2200 by communication—either wired or wireless. For example, processor 2300 may be a processor of a personal computer (PC) that receives the image information wirelessly from a camera of a portable (or fixed) unit. According to various embodiments of the invention, processor 2300 is connected to camera 2200—by way of communication (either wired or wireless), and possibly also mechanically (e.g. when located in a single unit).

Processor 2300 is configured to extract information of pixels (e.g. color information) from the multiple images based on an amount of hair information included in the pixels. That is, processor 2300 may be configured to retrieve pixel information from the multiple images (e.g. of pixels that correspond to each other in different images), to assess the amount of hair information in the various pixels (either independently or in comparison to one another), and in response to extract information of one or more of the corresponding pixels—based on the amount of hair information included in them—e.g. in order to determine pixel information for a corresponding pixel in a later generated synthetic image. It is noted that the term hair information indicates the amount of information of the pixel (or image) that originates from hair 3100.

Processor 2300 is further configured to generate, e.g. after all the required pixels have been extracted, a synthetic image of the body area, in which different pixels are extracted from different images of the multiple images. Thus, processor 2300 may generate a synthetic image in which areas that are hidden by hair 3100 in some of the multiple images are visible because the corresponding pixel information was taken from one or more pixels extracted from one or more images in which those areas were not hidden by the hair 3100. According to an embodiment of the invention, processor 2300 is configured to populate each pixel in the synthetic image with a corresponding pixel from one of the image frames, where the relevant pixel is as far as possible from the color of the hair (e.g., in the example above—the brightest pixel)

The blowing of air by air blower 2100 causes hair 3100 to move, and thus in different images, hair 3100 is located in different positions, and obstructs different portions of the body area. Experiments have showed that air blower 2100 may be signed and used to blow air so that camera 2200 may acquire the multiple images so that each area—except the roots of the hairs that may be located within body area 3000—of body area 3000 is cleared from hair 3100 in at least one image.

According to an embodiment of the invention, before being processed for the generation of the synthetic image, the multiple images may be aligned to one another (and/or otherwise adapted), so that corresponding pixels in various images may refer to the same portion of body area 3000. Such alignment may result in modification of one or more of the images, and/or by creation of conversion functions that enable to identify a pixel of a first image with the corresponding pixels of any of the other images. Various alignment algorithms are known in the art.

According to an embodiment of the invention, processor 2300 is further configured to process the multiple images for aligning the multiple images.

In most situations, all of hair 3100 is usually significantly darker or significantly brighter then the entire body area 3000. By way of example, if hair 3100 is dark, it would generally be darker than the skin of body area 3000 (even if the later is tanned or includes moles), and if hair 3100 is blond it may be much lighter than the skin of body area 3000.

In such situations, the brightness of the pixels extracted may be use to determine the hair information of the pixels. According to an embodiment of the invention, processor 2300 is further configured to select pixels according to the brightness of the corresponding pixels of the multiple images. For example, according to an embodiment of the invention, processor 2300 may be configured to select the brightest of all the corresponding pixels of the various images or to combine information from the N brightest pixels, and so forth (if hair 3100 is generally darker than body area 3000), and may be configured to select the darkest of all the corresponding pixels of the various images or to combine information from the N darkest pixels, and so forth (if hair 3100 is generally brighter than body area 3000).

According to an embodiment of the invention, processor 2300 is further configured to select the pixel having an extreme brightness value (i.e. either the minimal or the maximal), in response to hair darkness parameter (which indicates whether dark pixels or bright pixels are favored).

According to an embodiment of the invention, processor 2300 may be configured to process at least one of the multiple images for determining the hair darkness parameter. Thus, for example, if system 2000 may be used for both people with dark hair and those with bright hair, without manually selecting the hair darkness parameter.

It should be noted that both for a dark hair/light skin scenario and for a light hair/dark skin scenario, the majority of the pixels in the images will usually represent skin color and not hair color—and thus processor 2300 may conveniently be able to determine statistically what color threshold can be used to tell between clean skin areas and hair covered areas.

It is noted that other algorithms may be used for computing of pixel information and for the extraction of pixels based on hair information. For example, pattern identification parameters may be used for identification of hair 3100 (e.g. search for continuous lines of substantially uniform color), either in addition or instead of the brightness parameters. Other image processing parameters—many of which are known in the art—may be used for assessment of hair information in the various pixels.

It should be noted that comparing to prior art image-processing hair removal algorithms in the art that replace the pixels that include hair color with pixels that are meant to look like skin and which cannot and do not reveal the real skin under the hair—in the proposed solution real skin information is included in the synthetic image.

The removal in the hair in images used for melanoma screening is very important to patients with rich hair coverage, as the determination of visible features of the mole is very sensitive to high errors in calculation of the visual mole parameters such as contour roughness or color distribution.

analysis of the form of moles is very sensitive to minute details, such as edge details of the mole, symmetry analysis and so forth. Many moles are characterized by hairs sprouting from them or a located in a hairy part of the body, and such hairs may obstruct such detailed view of the mole. Therefore, the use of system 2000 enable to produce a synthetic images in which hair information (possibly except information pertaining to the roots of those hairs) is eliminated, and the mole is clearly visible and analyzable.

As aforementioned, air blower 2100 is used for moving hair 3100 of body area 3000 between different locations by blowing air toward hair 3100. it is noted that, according to an embodiment of the invention, air blower 2100 is further configured to blow air toward hair 3100 in various manners, e.g. different directions of wind, different strength of blowing, and so forth. Such modifications may be preplanned, and may be commanded by processor 2300 after analysis of some of the multiple images.

It is noted that various embodiments of system 2000 may have different utilization—e.g. in face recognition technologies, in beauty related fields, in medical uses, and so forth. Some of the fields in which systems 2000 may be useful are oncology and dermatology. For example, some embodiments of system 2000 may be used for generating synthetic images of moles, for assessment whether the mole is suspicious of cancer or not.

According to an embodiment of the invention, body area 3000 includes a mole. It is noted that the mole may be the subject of the multiple image, and be focused upon. This may be useful in systems that analyze moles, e.g. for detection of cancer. The focusing on the mole may be carried out automatically or manually (e.g. by a home-user, by a doctor, by a technician, and so forth).

It is noted that, according to an embodiment of the invention, the multiple images are cropped (e.g. by processor 2300, by a processor of camera 2200, and so forth) to include substantially the mole (or other significant feature—e.g. a scar, a tattoo, an ear) and the immediately surrounding area. According to an embodiment of the invention, the synthetic image may be constructed only information pertained to the cropped area.

According to an embodiment of the invention, system 2000 includes a processor (either processor 2300 or another processor, such as a dedicated processor) that is configured to analyze the synthetic image, and to provide a dermatologically significant result in response to a result of the analysis. In some other embodiments of the invention, such a processor may be configured to provide other types of medically significant results, e.g. oncologically significant results, and so forth.

It is noted that in the analysis of moles, any trend discovered in the evolution of a mole may be very significant as an alert on prognosis of cancer. According to an embodiment of the invention, imaging system 2000 is further adapted to repeat (e.g. periodically) the acquisition multiple images and the generating of the synthetic image at different days (e.g. every three days, every month), wherein a processor of the system is further configured to process a series of the synthetic images generated in the repetition (e.g. images taken every three days, every month) for recognizing a dermatologically significant trend. Such trends may be, for example, continuous growth of the mole, increasing asymmetry in the form of the mole, and so forth.

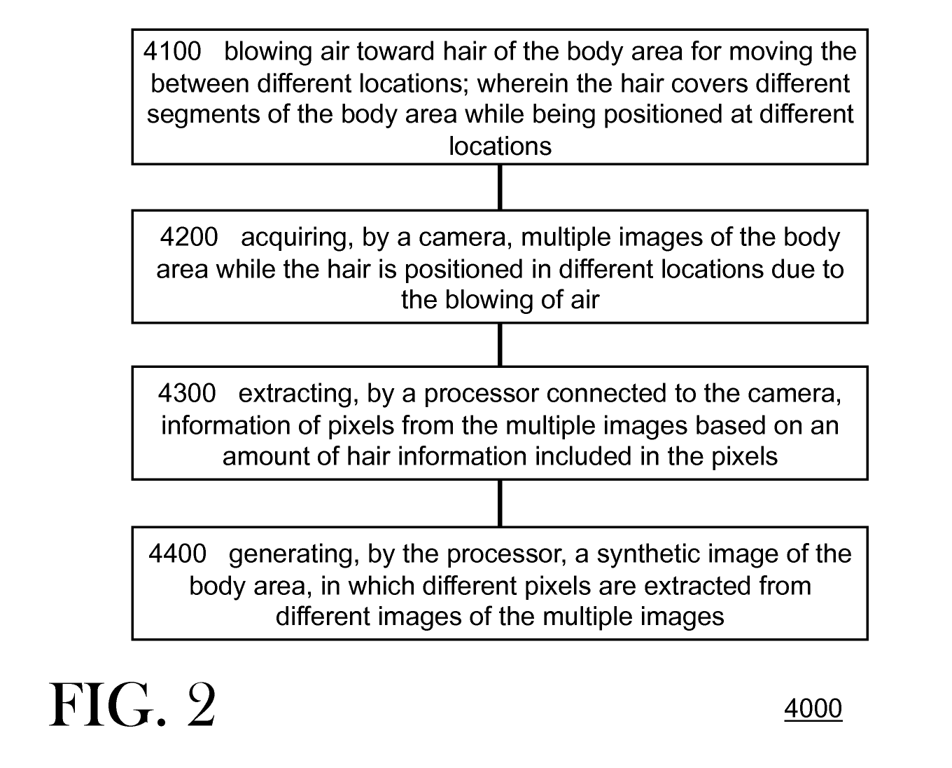

FIG. 2 illustrates method 4000 for imaging a body area, according to an embodiment of the invention. It is noted that embodiments of method 4000 may be made to implement the various embodiments of system 2000 (e.g. the embodiments discussed above), and vice versa. The terms used in the disclosure of method 4000 are substantially similar to those used above.

Method 4000 starts with stage 4100 of blowing air toward hair on the body area for moving the between different locations; wherein the hair covers different segments of the body area while being positioned at different locations. Referring to the examples set forth in the previous drawings, the blowing of stage 4100 may be carried out by air blower 2100 of system 2000.

Stage 4200 of method 4000 includes acquiring, by a camera, multiple images of the body area while the hair is positioned in different locations due to the blowing of air. It is noted that stage 4200 is at least partly concurrent to stage 4100 (even though some of the images may be acquired when no blowing is carried out). Referring to the examples set forth in the previous drawings, the acquiring of stage 4200 may be carried out by camera 2200 of system 2000.

Stage 4200 is followed by stage 4300 of extracting, by a processor connected to the camera, information of pixels from the multiple images based on an amount of hair information included in the pixels. Referring to the examples set forth in the previous drawings, the extracting of stage 4300 may be carried out by processor 2300 of system 2000.

Stage 4300 is followed by stage 4400 of generating, by the processor, a synthetic image of the body area, in which different pixels are extracted from different images of the multiple images. Referring to the examples set forth in the previous drawings, the generating of stage 4400 may be carried out by processor 2300 of system 2000. It is noted that stage 4400 may alternatively be carried out by a processor other than that of stage 4300.

According to an embodiment of the invention, method 4000 further includes selecting pixels by the processor according to the brightness of the corresponding pixels of the multiple images.

According to an embodiment of the invention, the selecting includes selecting by the processor the brightest of all the corresponding pixels.

According to an embodiment of the invention, the selecting includes selecting by the processor the pixel having an extreme brightness value, in response to hair darkness parameter.

According to an embodiment of the invention, the selecting is preceded by processing, by the processor, at least one of the multiple images and determining the hair darkness parameter.

Skin areas that are found to be too bright (close to the saturation level of the color) are assumed, according to an embodiment of the invention, to be flared by direct reflection from the light source, and may be discarded—even if they are likely to become the brightest pixels in the image.

According to an embodiment of the invention, the extracting is preceded by processing the multiple images, by the processor, for aligning the multiple images.

According to an embodiment of the invention, the acquiring includes acquiring, by the camera, multiple images of the body area that comprises a mole.

According to an embodiment of the invention, method 4000 further includes analyzing the synthetic image, and providing a dermatologically significant result in response to a result of the analysis.

According to an embodiment of the invention, method 4000 further includes repeating the stages of blowing air, acquiring multiple images, extracting information of pixels and generating a synthetic image at different days, wherein the method further comprises processing a series of the synthetic images generated in the repetition for recognizing a dermatologically significant trend.

It is noted that various embodiments of system 2000 and of method 4000 may be combined with the systems and methods described below. For example, the synthetic images generated by system 2000 may be used for analysis by one or more of the system discussed below, even if not explicitly elaborated in every instance.

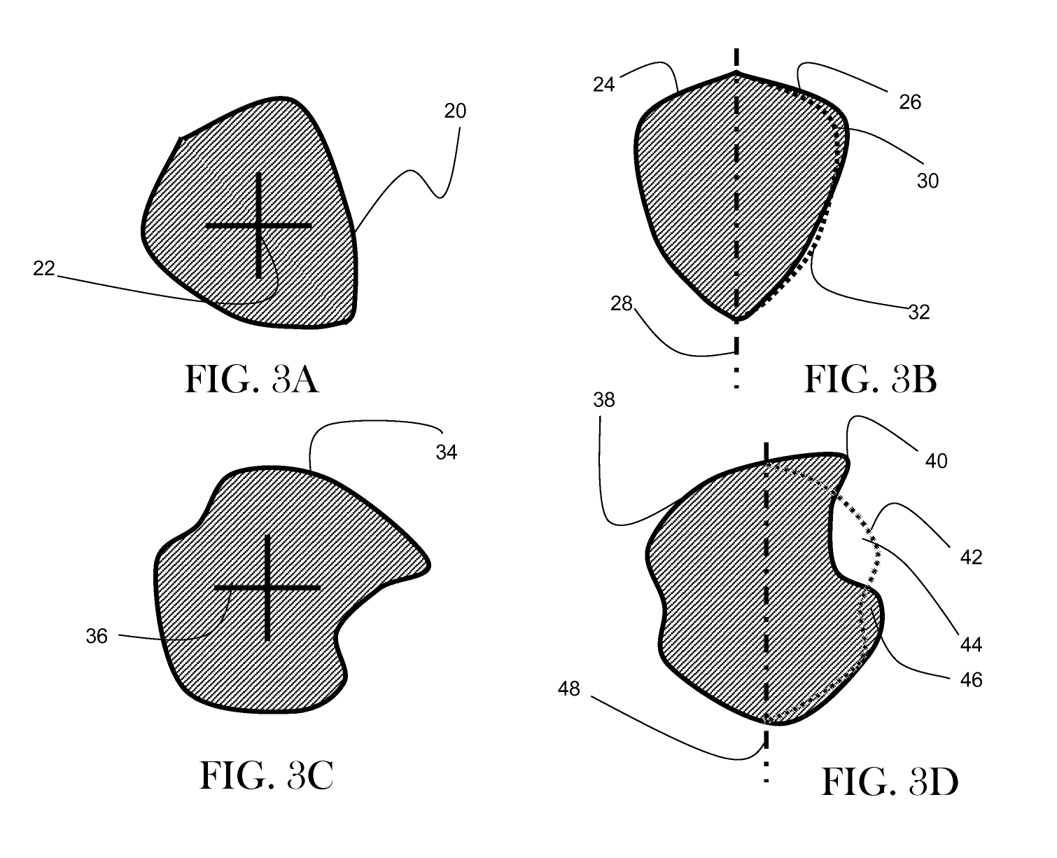

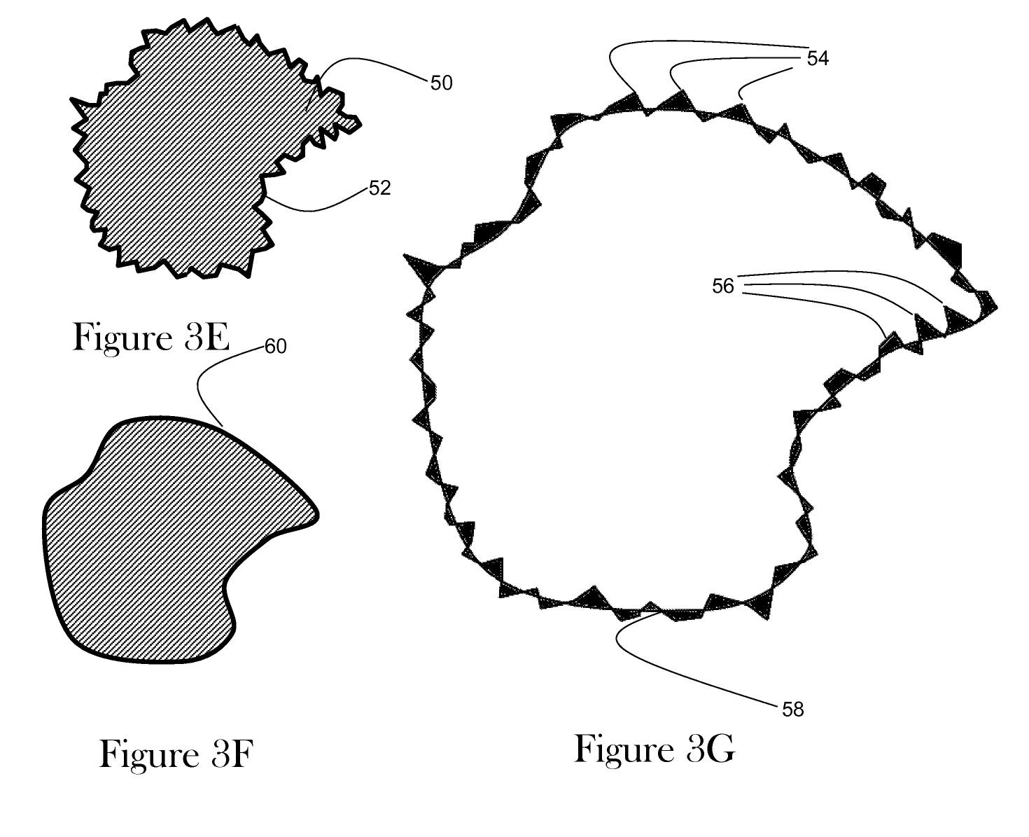

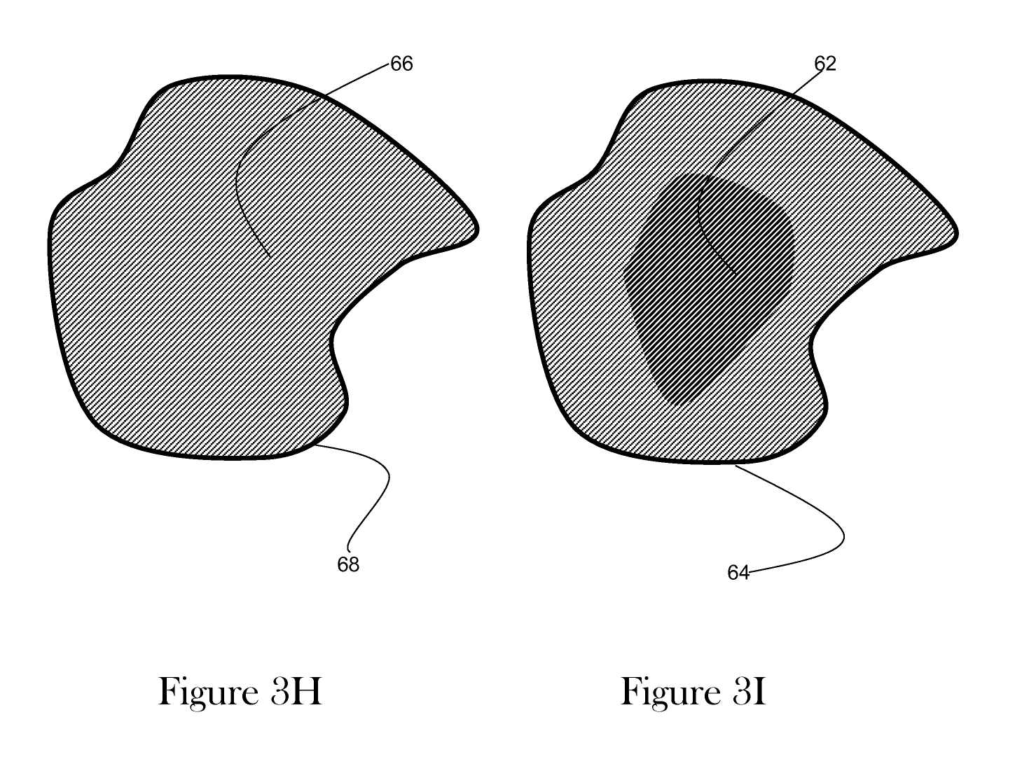

Referring to FIG. 3A-3D, that illustrates “symmetry” as may be implemented in various embodiments of the invention.

FIG. 3A shows a mole 20 and its center of mass 22, marked by a cross. There are well known equations in the art of geometry for calculating the location of the center of mass based on the contour of the mole.

FIG. 3B illustrates a mole 24 and a line of maximum symmetry 28. The line 28 is found by passing an arbitrary straight line through the center of mass and calculating the symmetry around this line (flipping the contour from one side of this line to the other side of this line and calculating the area captured between the original part of the contour 26 and the flipped part of the contour, part of which 30 lays inside the stationary part 26, and part of which 32 lays outside the stationary part 26. If the shape is totally symmetric around the line, the captured area will be zero. If the shape is non-symmetric around this line the captured area will differ from zero. The total area is indicative of the level of symmetry around this line. Then, the algorithm rotates the line 28 about the center of mass in small angular increments, and keeps calculating the symmetry, seeking the direction of minimum area, indicative of maximum symmetry. The area at maximum symmetry will be outputted as the symmetry parameter of the mole.

FIG. 3C illustrates a different mole 34 with a center of mass 36, that is clearly less symmetrical than the mole of FIG. 3A.

FIG. 3D illustrates a captured area of mole 38 around the line of maximum symmetry 48, with flipped contour 42 over stationary contour 40, with internally captured area 44 and externally capture area 46. It should be noted that the captured area in FIG. 3D is much larger than the captures area of FIG. 3B, indicating a much less symmetric mole.

Referring to FIG. 3E-3G that illustrates an algorithm for calculating the roughness (or—irregularity) of the border of a mole, according to an embodiment of the invention.

FIG. 3E illustrates a mole 50 with a rough border 52,

FIG. 3F illustrates the same mole, where the border 60 is smoothened mathematically—typically by approximation to a low degree curve such as disclosed in relation to FIG. 2.5 in the article “ON THE THEORY OF PLANAR SHAPE” by Lisan et. Al, published by Multi Scale Model, Simul, vol. 1, no. 1, pp. 1-24, 2002

FIG. 3G illustrates an overlay of FIG. 3F over FIG. 3E, and the area captured between the original shape of 3E (54, 56) and the smoothened shape of 3F. The total area will be defined as the roughness parameter of the mole.

Referring to FIGS. 3H and 3I that illustrate color variation within the mole. FIG. 3H illustrates a relatively uniform 66 color of the mole 68. FIG. 3I illustrates a relatively varying 62 color of the mole 64. The distribution of the hue of the mole area is calculated, and the variance of that distribution is a clear indication to the variance and can be used as the color variance parameter.

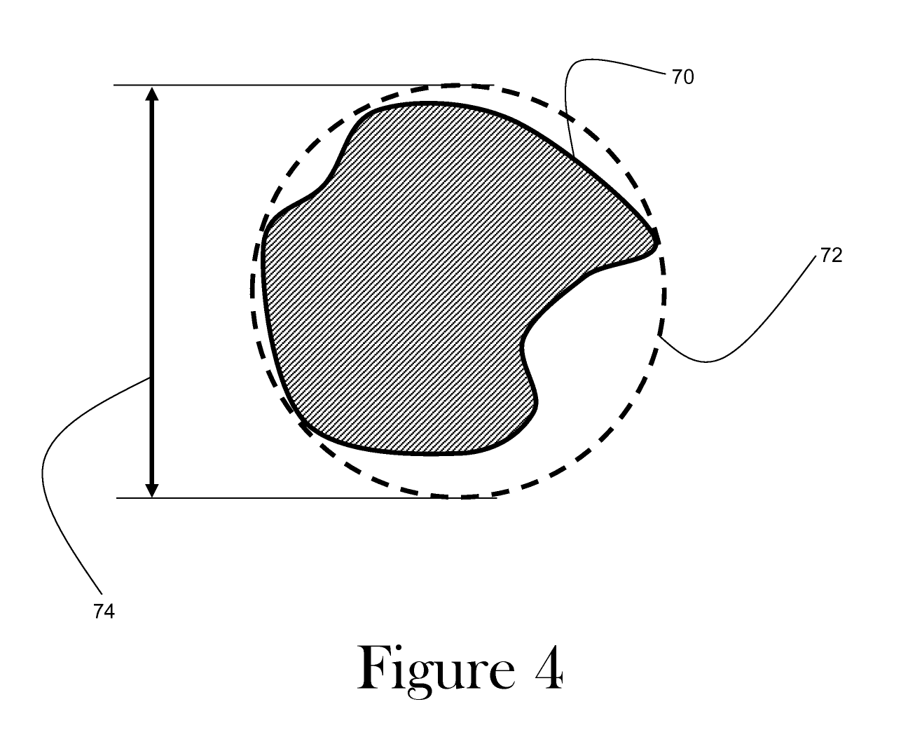

Referring to FIG. 4, that illustrates determination of a diameter of a mole 70, according to an embodiment of the invention. The algorithm finds the smallest circle 72 that bounds the mole, and uses its diameter 74 as the diameter of the mole.

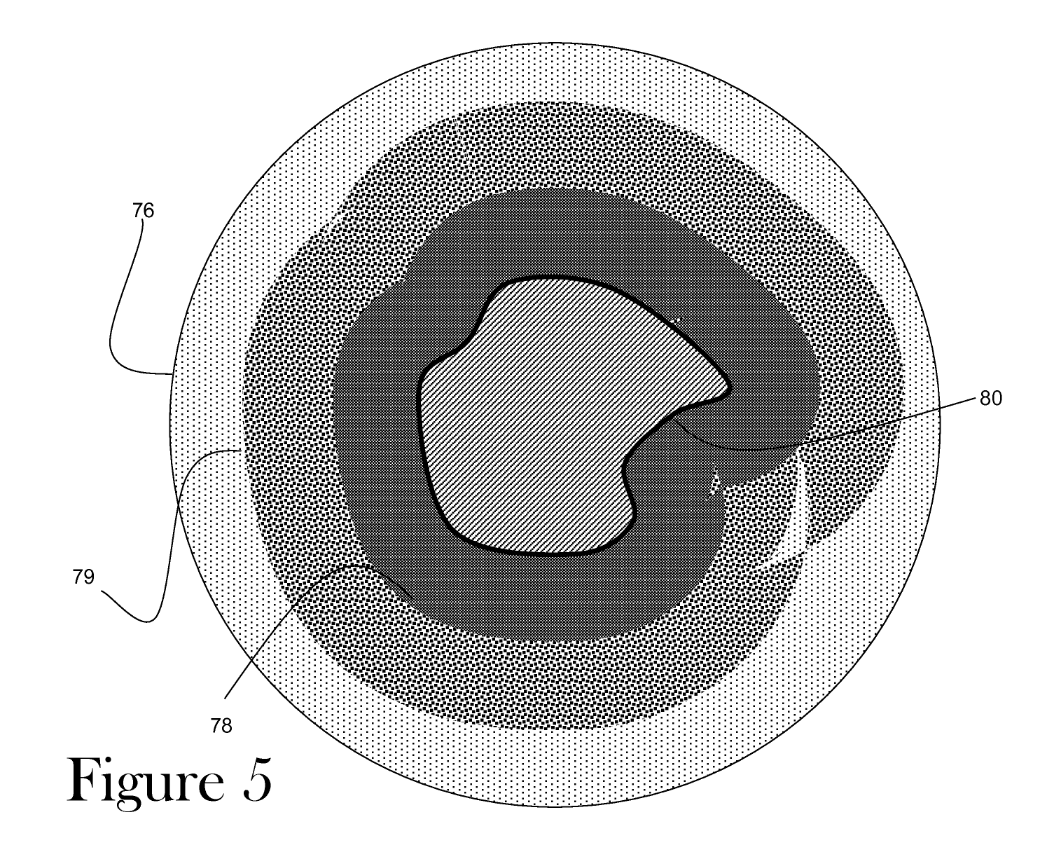

Referring to FIG. 5 that exemplifies mole redness, which is a response of body tissues to mole, a reddening of the skin. A mole 80 is shown. Its diameter is calculated as disclosed in relation to FIG. 4. A margin with a given width (typically ⅓ of the diameter) 78 is drawn around the mole. A second, external margin 79, typically of the same width as margin 78, is drawn. The average red content of the margins 78 and 79 is calculated, and the ratio between them is an indication of the gradient in the skin color outside the mole. This will serve as the “redness parameter”. The parameter is normalized to the general tan of the skin 76 so that the increase of redness due to temporary tanning is compensated.

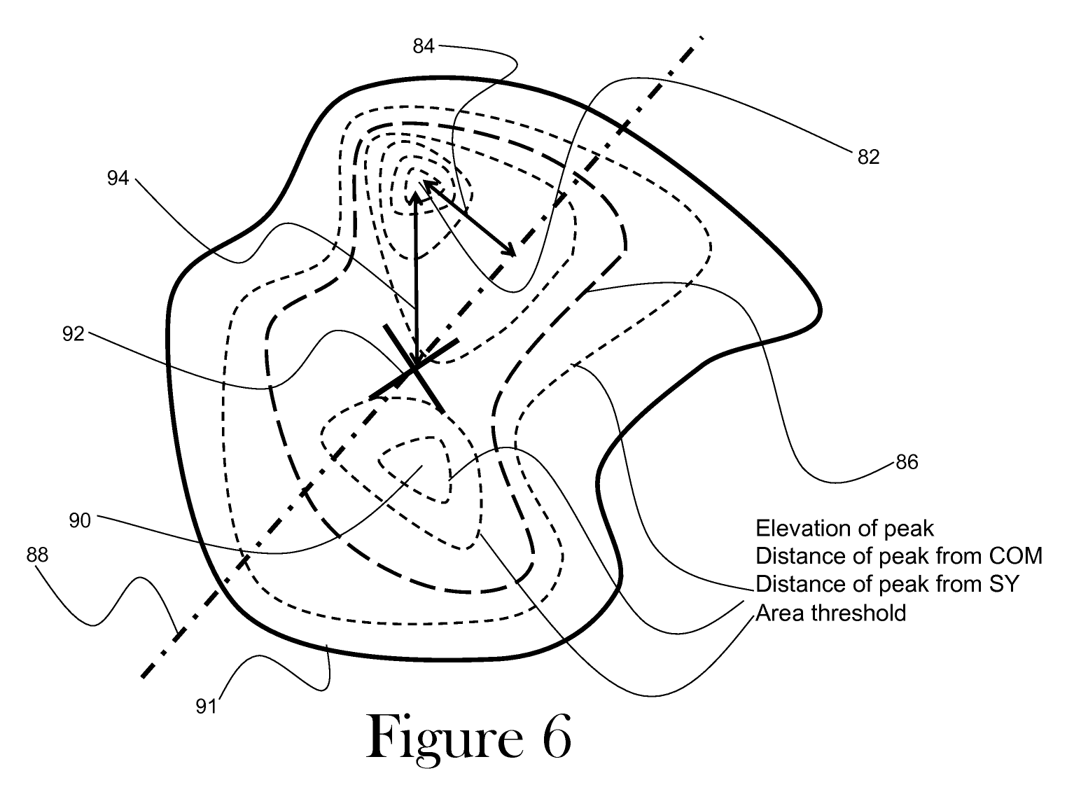

Referring to FIG. 6, which illustrates several parameters of mole topography which may be utilized in different embodiments of the invention.

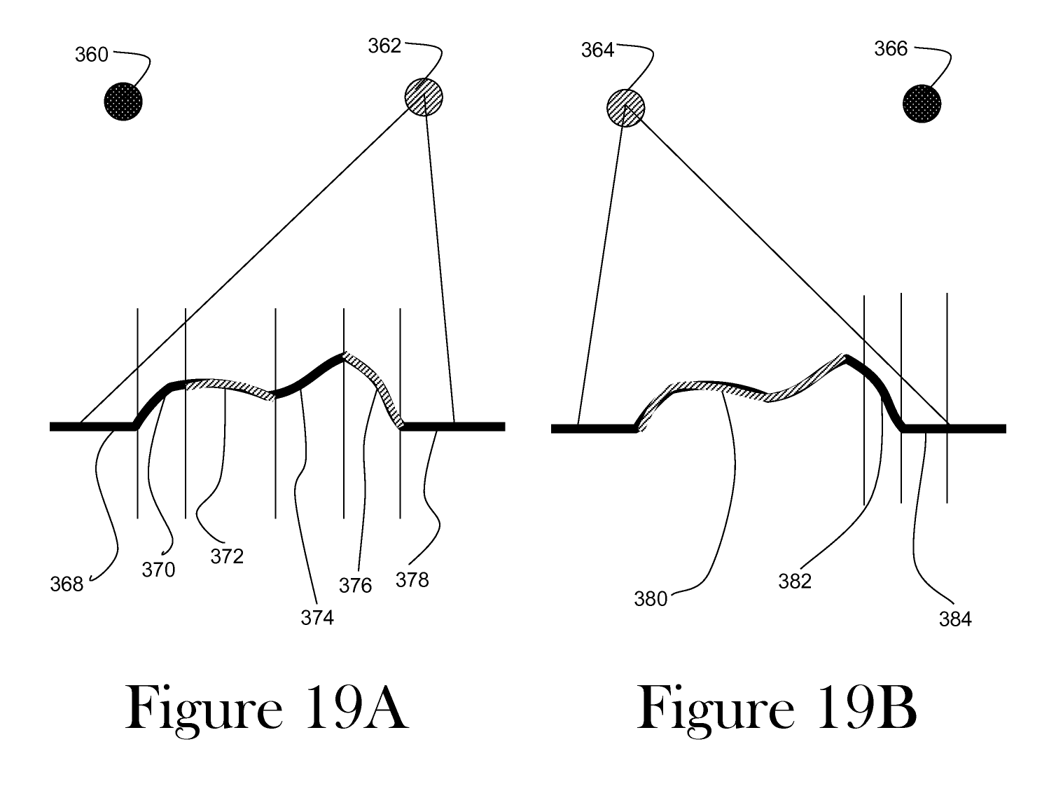

In preparations to the topography parameterization, the algorithm calculates the center of mass 92 (as disclosed in relation to FIG. 3A) and axis of maximum symmetry 88 (as described in relation to FIG. 3B) of the mole 91. The algorithm uses the DTM data of the mole (DTM and its measurement will be explained below in FIGS. 19A and 19B) to identify the local peaks of the mole (82, 90) and their relative elevation.

The topography of the mole will then be represented by the following parameters:

The elevation of the highest peak above the average elevation of the mole. The distance 84 between the highest peak and the axis of maximum symmetry.

The distance 94 between the highest peak and the center of mass of the mole

The area of the mole that is below or above a threshold 86 of a given fraction of the highest peak. This parameter will be very large for convex moles, and much smaller for moles that have concave areas.

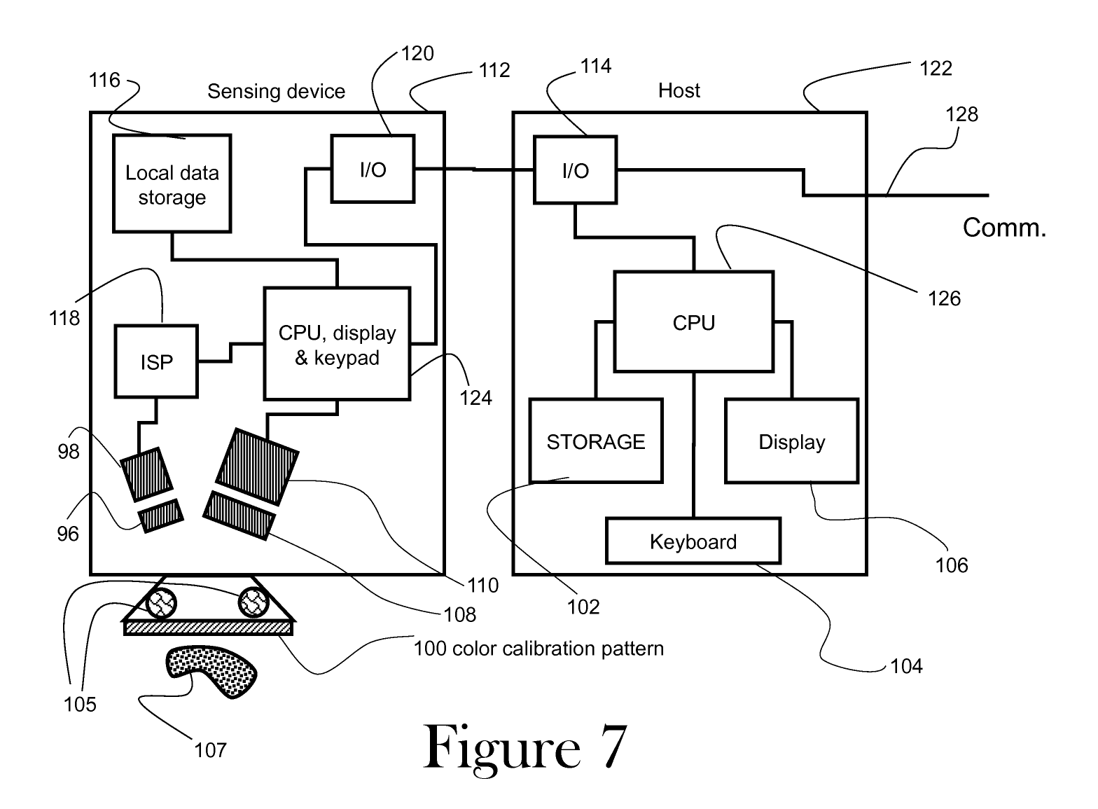

Referring to FIG. 7, that illustrates a block diagram of a system, according to an embodiment of the invention. A sensing device 112 is connected to a host computer 122. The sensing device 112 can be connected to the host through an IO block 120 (in the device) and 114 (in the host) via a cable, a wireless connection, an infrared link or any other means of connecting a peripheral to a host.

The sensing device contains a local data storage 116, such as a flash drive or an embedded hard disk drive for storing images when the sensing device is off-line, and a conventional processing components such as a CPU, keypad and a display (collectively—124).

A focused grid pattern of light is generated by a light source 110 and an optical lens 108, such as diffractive optical elements (DOEs) made by StockerYale Canada Company, Montreal, Quebec, Canada and is projected onto the mole area 107.

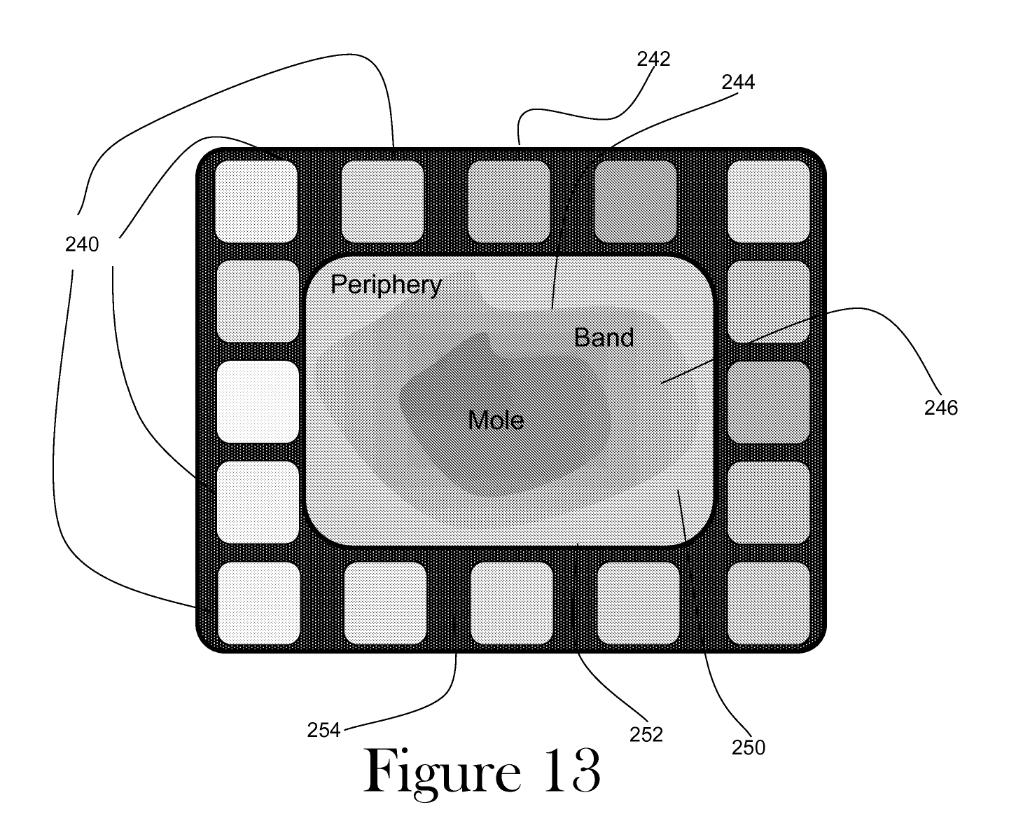

A camera 98 with a lens 96 is looking at the mole area 107 covering also a color calibration pattern 100 such as described in detail in relation to FIG. 13. Two or more controllable point light sources 105 illuminate the scene.

When the system needs to take an image for a two dimensional analysis, such as border irregularity or color, the point light sources 105 that can be standard while LED's, are switched on to illuminate the scene. The camera 98 is focused on the mole area 107, capturing the mole and the calibration ring. The calibration the image can be calibrated using the calibration ring as will be explained in FIG. 13 below. The image is processed into a standard image file by the image signal processor 118 and is stored in the local data storage 116.

When the system needs to take an image for a three dimensions analysis, namely—topography, it can use one of two methods for gaining information about the topography of the mole area.

In one method, the point light sources 105 are sequentially turned on and off, so that the image contains illuminated and shadowed areas. An analysis of the local brightness of the image will provide an indication on the topography.

In another method, a grid pattern is projected on the mole form an angle that is different from the angle of the imaging, so that an active stereo effect is created. It is noted that Active stereo emerges as an alternative approach to the traditional use of two cameras. The word “active” here signifies that energy is projected into the environment. In an active stereo vision system, one of the cameras is replaced with a projector or a laser unit, which projects onto the object of interest a sheet of light at a time (or multiple sheets of light simultaneously). The idea is that once the perspective projection matrix of the camera and the equations of the planes containing the sheets of light relative to a global coordinate frame are computed from calibration, the triangulation for computing the 3-D coordinates of object points simply involves finding the intersection of a ray (from the camera) and a plane (from the sheet of light of the projector or laser).

The grid pattern appears to the camera to be distorted, and the distorted lines can be analyzed to produce the topography of the mole. The active stereo method of topography analysis is well known in the art and is described in:

Alexander, B. F. & Ng, K. C. (1987), 3D Shape Measurement by Active Triangulation using an Array of Coded Light Stripes, in ‘Proc. SPIE Vol 850: Optics, Illumination and Image Sensing for Machine Vision II’, Vol. 850, pp. 199-209.

The Evolving, Distributed, Non-Proprietary, On-Line Compendium of Computer Vision by Robert B. Fisher, Editor, School of Informatics, University of Edinburgh, Scotland, UK

The host 122 contains a CPU 126, a display 106, a keyboard 104, and storage 102. The host is reading the images from the sensing device when the sensing device is in communication with the host. Image processing algorithms that will be described hereinbelow determine the parameters of the mole, a pattern recognition program identifies the mole against the list of known moles of the patient, and an historical analysis program identifies deviations of the mole by comparing the current image to its recent history. Alerts on systematic deviation of a mole from its known parameters are generated and reported via communication link 128.

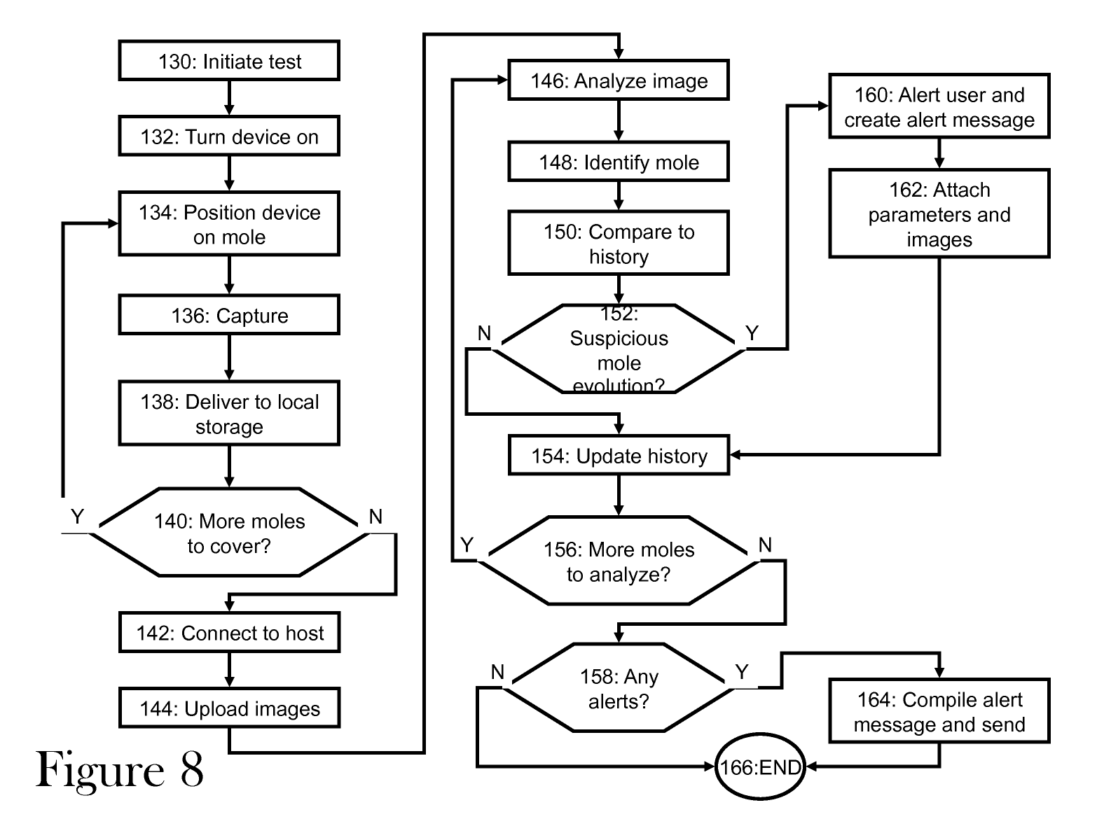

Referring to FIG. 8 that illustrate a general flow chart of the operation of the system, according to an embodiment of the invention.

When the user initiates 130 a test and turns the device on 132, he positions 134 the device above one of the moles to be examined The user then captures 136 an image, and the image is stored 138 locally. If there are more moles to cover, then the user repeats 134. If this was the last mole to be examines, the user connects 142 the sensing device to the host, and uploads 144 the images to the host. The host then analyzes146 the image and retrieves the mole parameters, then identifies 148 the mole and compares 150 the current parameters t the history of parameters of that mole. If the comparison generates 152 a suspicion that the mole is changing, the system alerts 160 the user and creates an alert message to be sent to a remote care giver, attaching 162 the relevant parameters, and also updates 154 the history of the mole.

If the mole parameters do not show a deviation, the system updates 154 the mole history. If there are additional moles to be analyzed, the system goes back to analyze 146 the next mole images.

When all the moles have been analyzed, the system checks 158 if there are alerts and if so—compiles 164 a summary of the alert messages and sends them via conventional communication means to a care giver—typically the physician in the clinic.

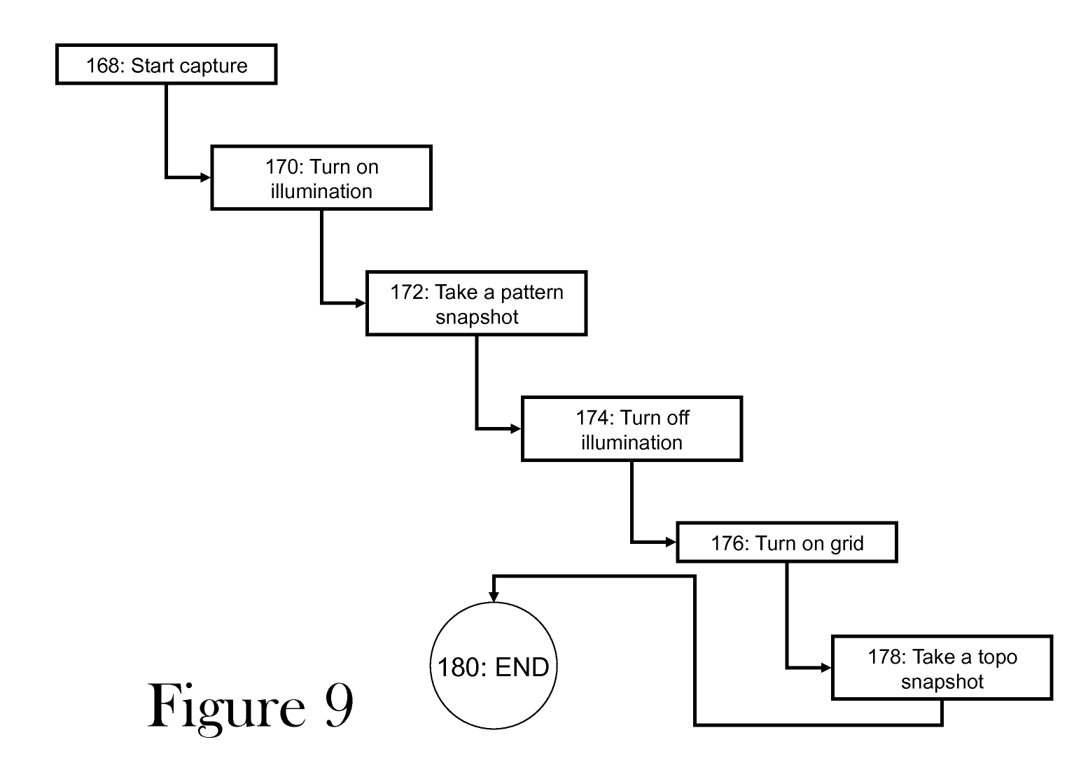

Referring to FIG. 9, that illustrate a flowchart of the capture process, according to an embodiment of the invention. The user starts 168 the capture process, the controller in the sending device turns on the flood illumination (the LED's) 170, than takes 172 a snapshot of the mole area for extracting all the two-dimensional parameters, then system turns off 174 the flood illumination and turns on 176 the grid illumination and captures 178 a second image for the three dimensional parameters.

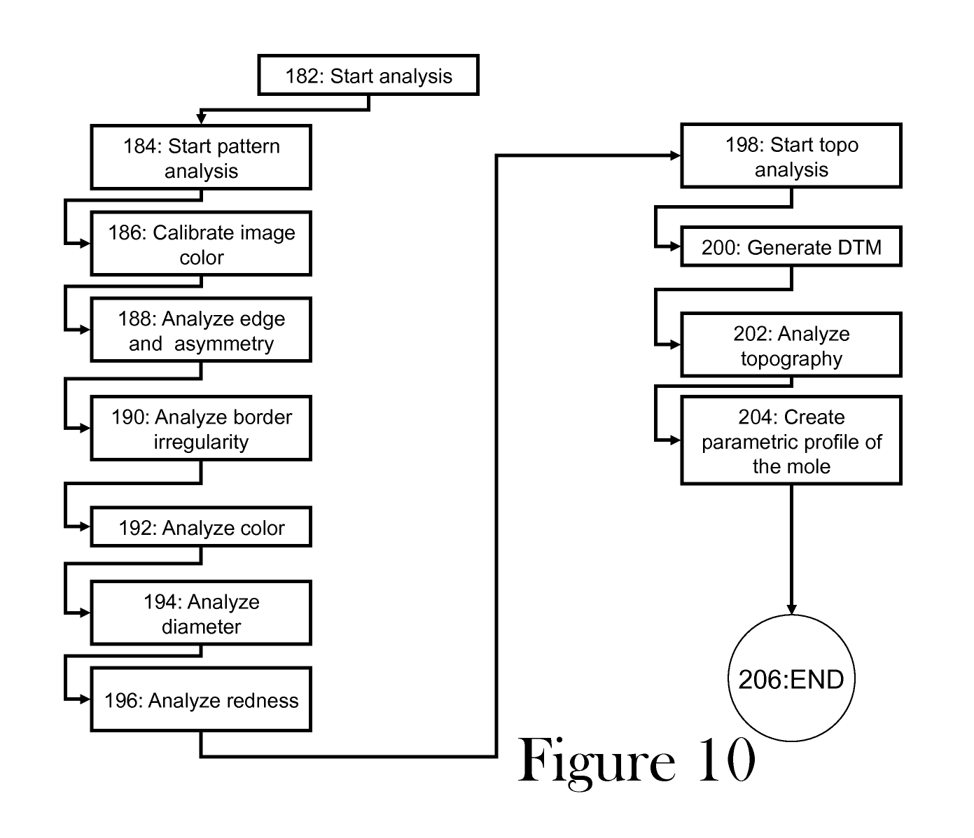

Referring to FIG. 10 that illustrate a flowchart of the image analysis process according to an embodiment of the invention, which is a “blowup” of the reference number 146 in FIG. 8.

The analysis starts 182 with a pattern analysis 184. The first step is calibration of the image colors 186 in accordance with the colors of the calibration ring that are known to the system and is apart of the image. This compensates for any color deviations that are due to non uniform illumination, deviations in sensitivity of the camera etc.

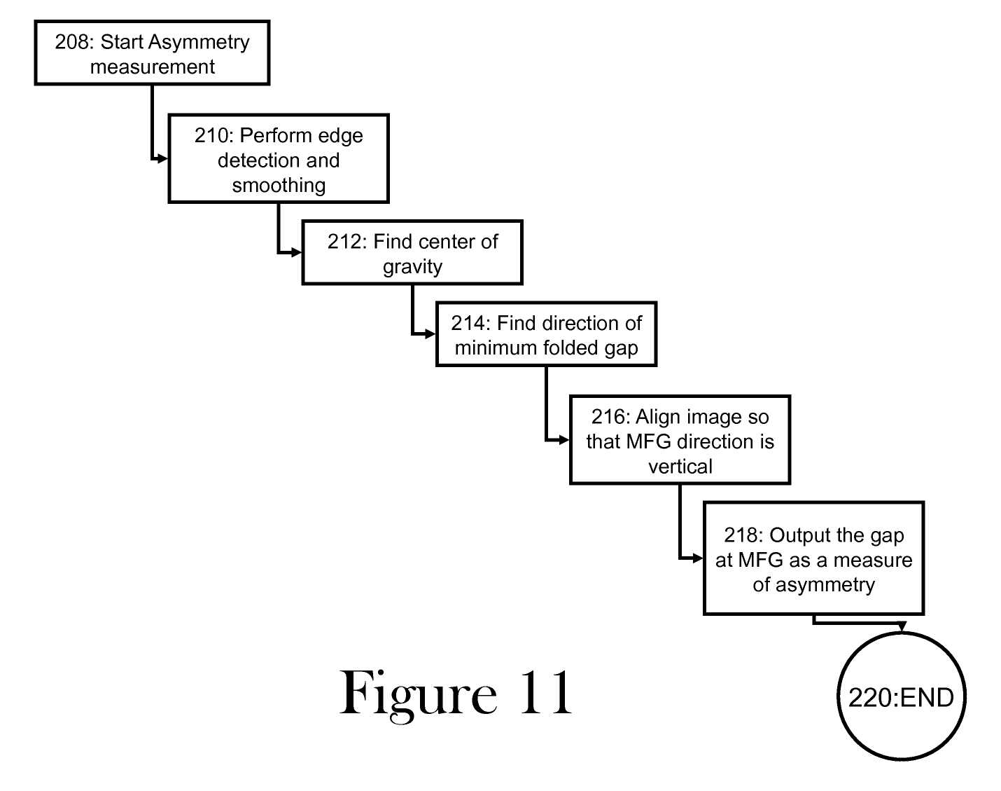

Following the color calibration, the system detects the edge of the mole and analyses 188 the asymmetry of the mole. This process is described in detail in relation to FIG. 11.

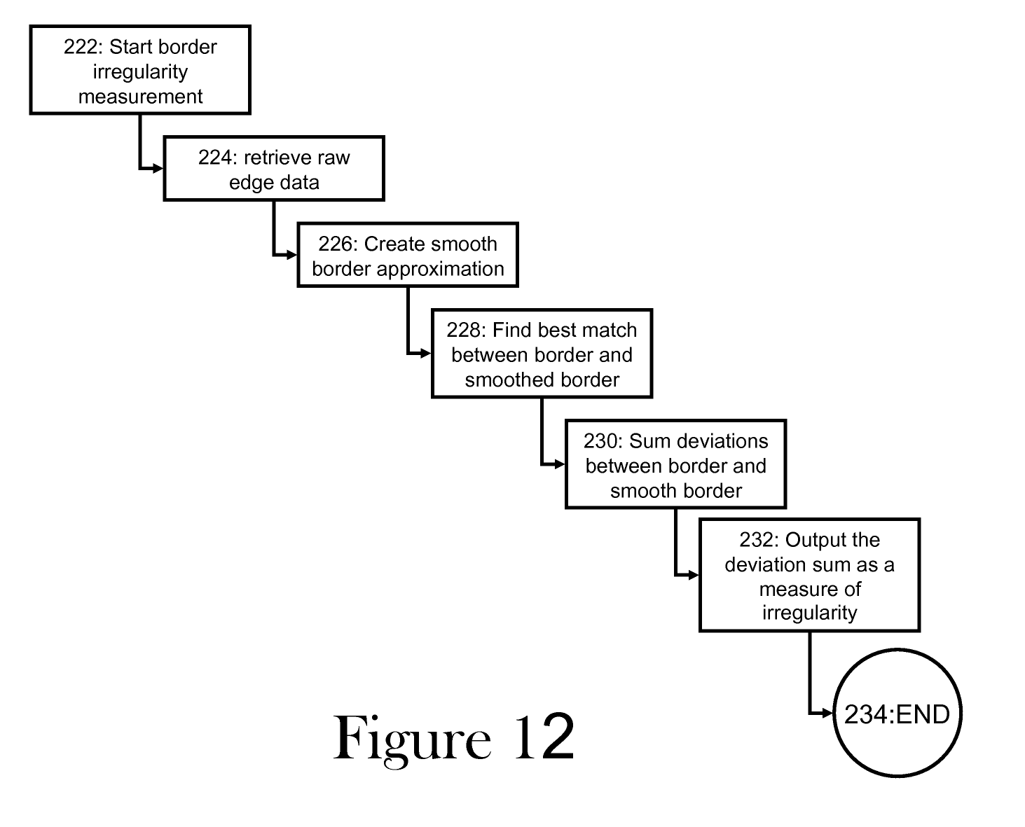

The system then analyses 190 the border irregularity. The details are given in relation to FIG. 12.

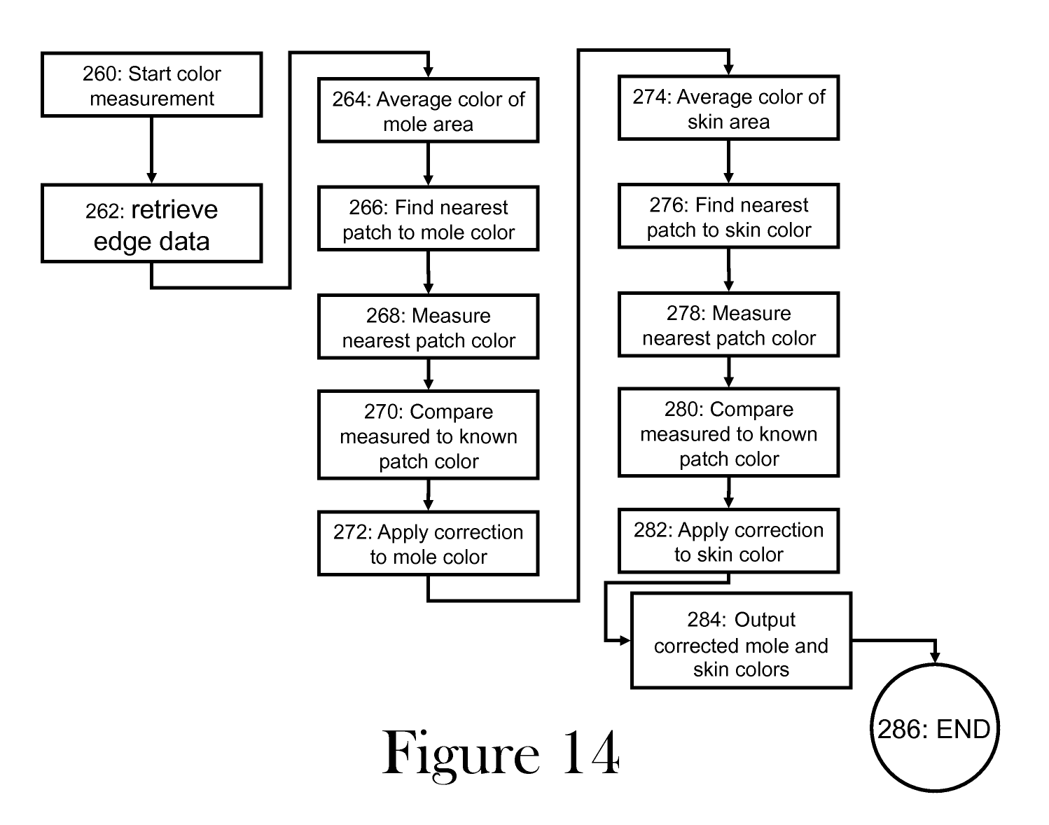

The system then analyses 192 the color of the mole. The details are given in relation to FIG. 14.

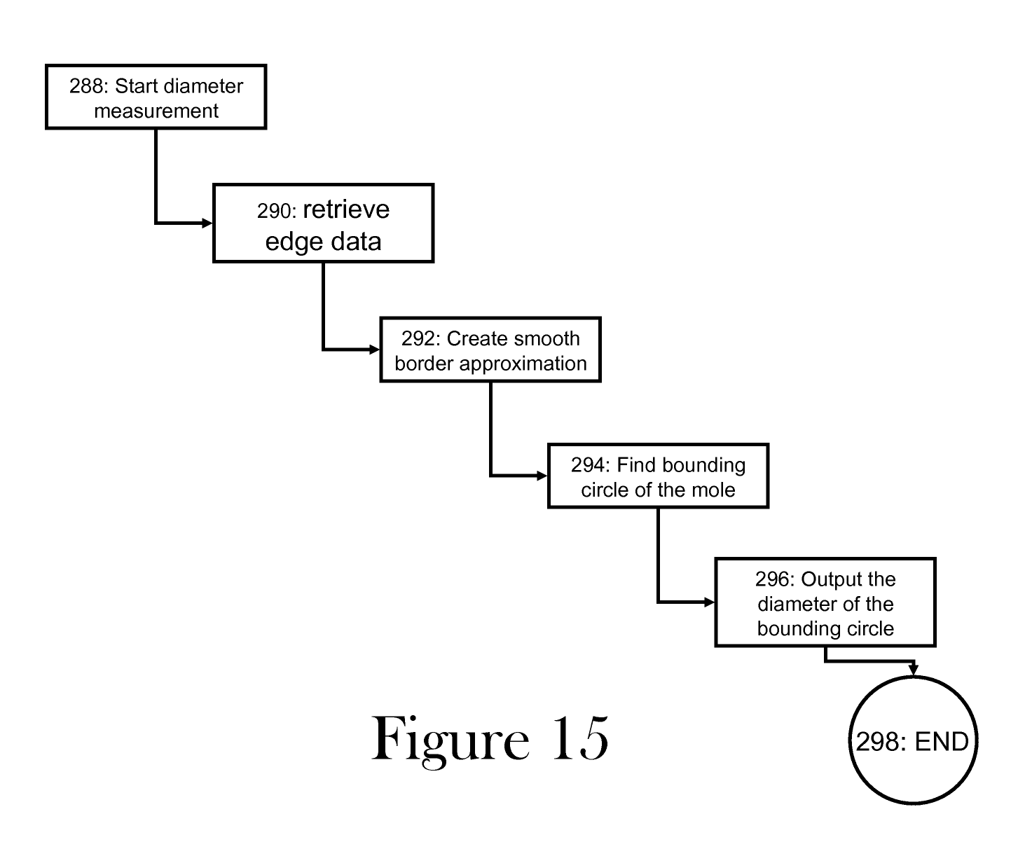

The system then analyses 194 the diameter of the mole. The details are given in relation to FIG. 15.

The system then analyses 196 the redness of the mole, as defined in detail in relation to FIG. 5.

The system then analyses 198 the topography of the mole.

The first step is to generate 200, from the raw stereometry data, a digital terrain model of the mole, using methods that are described in the aforementioned sources.

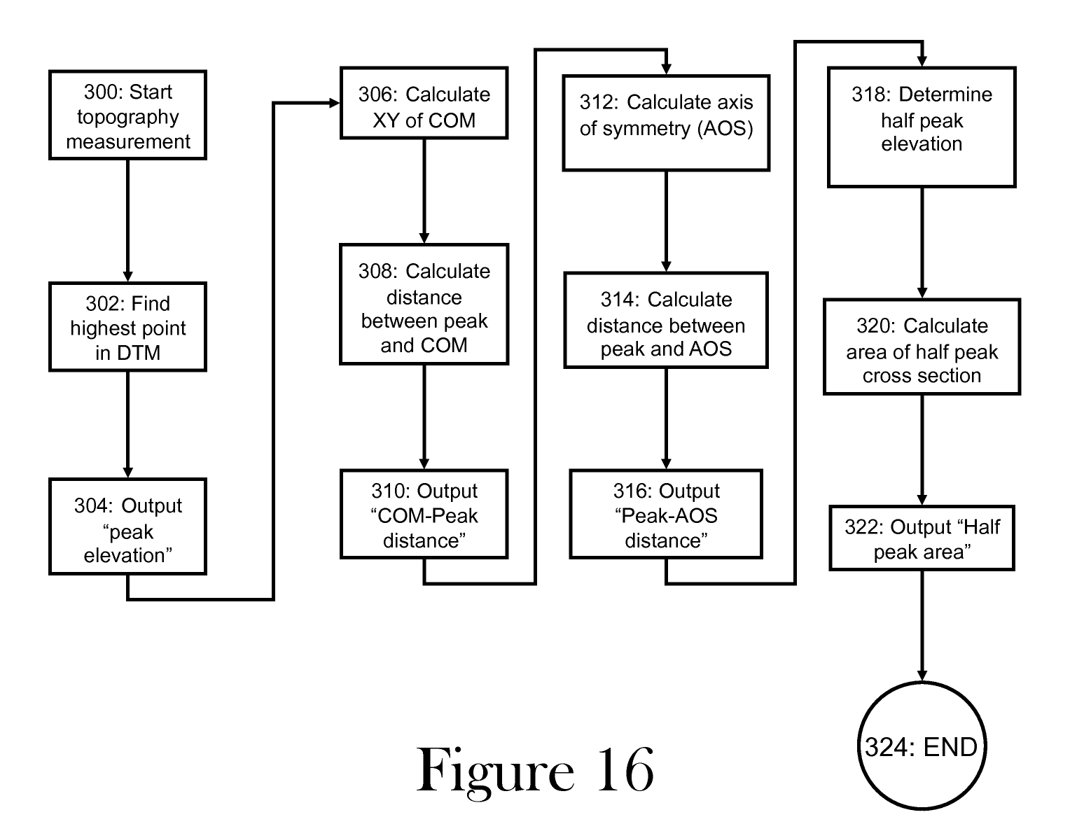

The system analyses 202 the DTM, to retrieve topographic parameters as described in relation to FIG. 16.

The system then concludes 204 the analysis of the two dimensional and three dimensional parameters of the mole, producing a parametric description that can be stored and compared to parametric descriptions of other moles and of the same mole at other times.

Referring to FIG. 11, that illustrates analysis of asymmetry 208, according to an embodiment of the invention.

The system performs edge detection 210—using one of the many edge detection algorithms known in the art—such as described in the article “Algorithmic reproduction of asymmetry and border cut-off parameters according to the ABCD rule for dermoscopy” by Pellacanni et al, in Journal of the European Academy of Dermatology and Venereology Vol. 20 Issue 10 Page 1214 November 2006. The detected edge is then smoothened—typically by approximation to a polygon of a relatively low degree, as the use of the edge does not typically require precise trajectory.

The next step is to find 212 the “center of gravity” of the mole that is done any of the common algorithms knows in the art for determination of center of mass of a two dimensional object of constant density.

Then the system finds 214 the direction of the straight line passing through the center of mass, around which the mole shows the maximum symmetry. Symmetry can be estimated by the area captured between the two halves of the mole, when folded about the axis.

The value of this measure, when minimum, is taken to describe the asymmetry of the mole. Other measures, that give minimum value when the mole is symmetrical, can be used.

The system then registers 216 the image so that the axis of symmetry (for minimum asymmetry) is directed along the Y axis (or any other predetermined direction, e.g. the X axis). This is done in order to normalize all the images of the same mole so that they can be compared.

It should be noted that the above mentioned algorithm may make a mistake of 180 in positioning the mole. This mistake can be avoided by selecting the one out of the two alternative positions, so that the center of gravity will be on the same side of the median of the axis of symmetry.

The system then outputs 218 the value of the asymmetry of the mole as one of the parameters.

Referring to FIG. 12, that illustrates estimation 222 of border irregularity, according to an embodiment of the invention.

The system retrieves 224 the raw (not the smoothened) edge detection data of the mole. The system then smoothens 226 the edge significantly, and tries to match 228 the smoothened and raw borders to have best fit. Real life borders of a mole will not be identical to their smoothened shape, and the difference between the two is a measure of the irregularity of the edge. The system measures 230 the area captured between the two curves, and this area is outputted 232 as the mole edge irregularity. This value can optionally be normalized to the diameter of the mole (to be described later) in order to compensate for size of the mole.

Referring to FIG. 13, that illustrates color calibration process, according to an embodiment of the invention.

The camera (not shown) has a field of view 242 that is partially blocked by a floor 254, made of a typically opaque and dark surface such as a dark plastic. The floor has a large opening 252 in its center—large enough to contain the largest possible mole 244 and some of its skin environment 250. The camera is focused to the floor. If the floor is pressed against the skin, the skin and its content are also in focus. A set of color calibration tags 240, representing all the ordinary colors of skin and mole, are arranged on the floor around the opening. They are also captured by the camera and are also in focus. The tags and the skin are illuminated by the same light source, and therefore, the tags (the color of which does not change and is pre-known) can serve as calibration areas for the image. The calibration can be done, for example, as follows: Inspect an area on the skin or mole. Find the tag that is most similar in color to the inspected area. Measure the Red, Green and Blue components of the color of the imaged tag, compare them to the nominal RGB values of the tag, extract the offsets in R, G and B between the nominal tag and the imaged tag, and apply the same offsets to the imaged skin.

If the mole is surrounded by an area of redness 246, which is an important symptom in melanoma diagnosis, the redness area will also be imaged and analyzed.

Referring to FIG. 14, that illustrates a flowchart of color determination, according to an embodiment of the invention.

The edge data is retrieved 262 from the mole contour analysis as explained in FIG. 1. The average color of the area within the mole contour is calculated 264. The calibration patches in the image (see FIG. 13) are identified 266 and their average color is calculated 268. The system them finds 270 the patch whose average color is nearest to the average color of the mole. As the measured color of the calibration patch is typically different than the reference color as stored in the system, the difference in color, in terms of hue and saturation, are applied 272 to the color of the mole, thus calibrating the mole color.

The system then averages 274 the color of all or some of the skin area outside the mole contour, finds 276 the nearest patch in terms of the average color and measures 278 the color of that patch, and then compares 280 and calibrates 282 the colors of the skin as described hereinabove.

The system then outputs 284 the average color of the mole and the average color of the surrounding skin—as two additional parameters of the mole.

The redness parameter of the mole can also be measured in a similar way—the skin that is external to the mole is divided into two areas—the inner area is a closed band around the mole—typically 4 mm wide. The outer area is the rest of the skin in the field of view. Now the system deals with three separate areas—the mole, the band and the periphery, and measures and reports the color of each using the above mentioned method. Redness is defined as the difference between the color of the band and the color of the periphery.

Referring to FIG. 15, that illustrates a flowchart of diameter measurement process according to an embodiment of the invention.

The system retrieves 290 the edge data of the mole as explained in FIG. 1 and creates 292 a smooth border approximation of the mole. The system finds 294 a tight bounding circle around the mole. The diameter of this circle is output 296 as the diameter of the mole as an additional parameter of the mole.

Referring to FIG. 16, that illustrate a flowchart of the topography measurement of the mole per the process described in relation to FIG. 6, according to an embodiment of the invention. The explanation that follows assumes that the digital terrain model (DTM) of the mole area is available.

The system finds 302 the highest peak in within the mole contour. The system outputs 304 this peak elevation as a mole parameter. The system then calculates 306 the XY coordinates of the center of mass (COM) of the mole. The system calculates 308 the distance between the peak and the COM, and outputs 310 this COM-Peak distance as a parameter. The system calculates 312 the axis of symmetry (AOS) of the mole. The system then calculates 314 the distance between the peak and the AOS line, and outputs 316 the Peak-AOS distance as a parameter.

The system then determines the half peak elevation 318 and calculates 320 the area of half peak cross-section. The system then outputs 322 the half-peak area as a parameter.

These parameters will be collectively used as a “profile” of the mole topography. Clearly, they are not sensitive to rotation, shift or vertical compression of the mole.

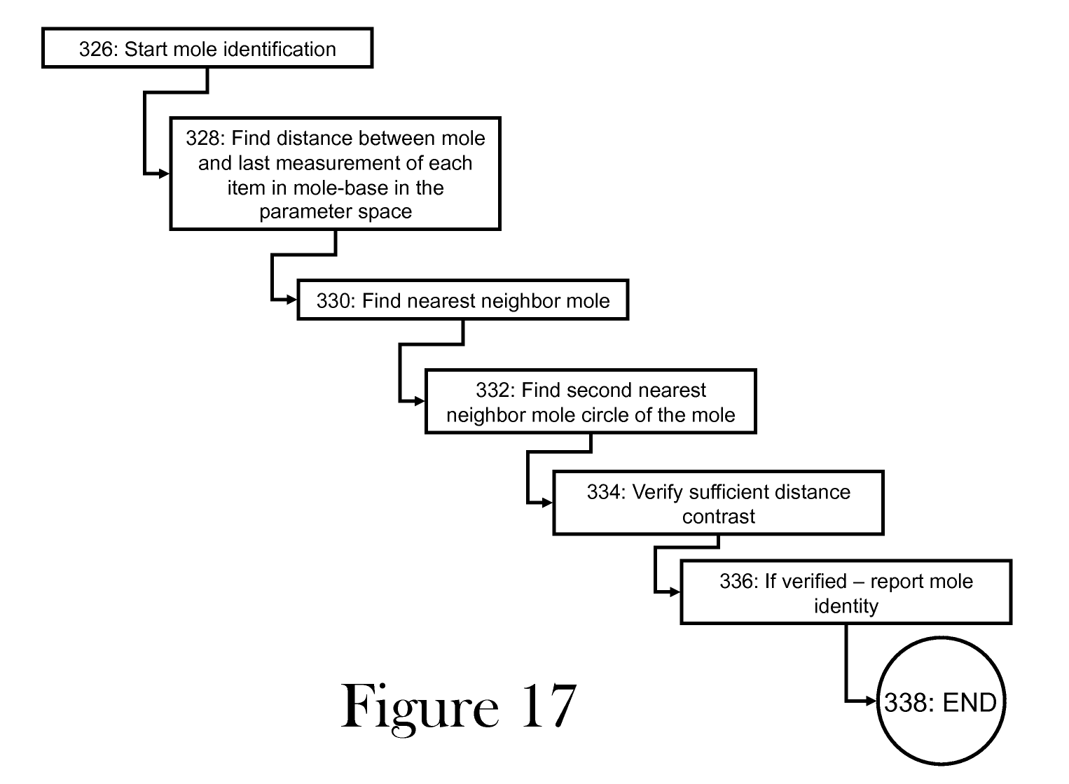

Referring to FIG. 17, that illustrate a flowchart of the identification of the captured mole, among the known moles of the user, according to an embodiment of the invention.

The system retrieves vectors of numbers that represent the last measurement of each of the known moles of the patient, from a mole-database stored in a local or a remote storage device—typically—a database in the hard disk of a personal computer or a local storage in the hand-held capturing device.

The system then calculates 328 a distance measure between the newly captured vector of parameters and each of these “historical” vectors.

The distance function between two vectors is found using conventional pattern recognition, feature extraction and clustering methods, well known in the art of statistical pattern recognition, using a large number of measured vectors of a large number of moles of a large number of patients, and optimizing the weights of each parameters so that all vectors representing the same mole result in small distances from each other, while different moles result in large distance between vectors.

The system then finds 330 the nearest neighbor mole among the known moles of the user to the newly captured mole. The system then finds 332 the second nearest neighbor, and verifies 334 that the contrast between the two distances is sufficient to avoid false detection.

If verified 336, the system reports a mole identity and has thus automatically detected the measured mole. The algorithm described here relives the user from the need to manual identify the moles or to scan them in a prescribed order. The user can visit the moles at any desired order.

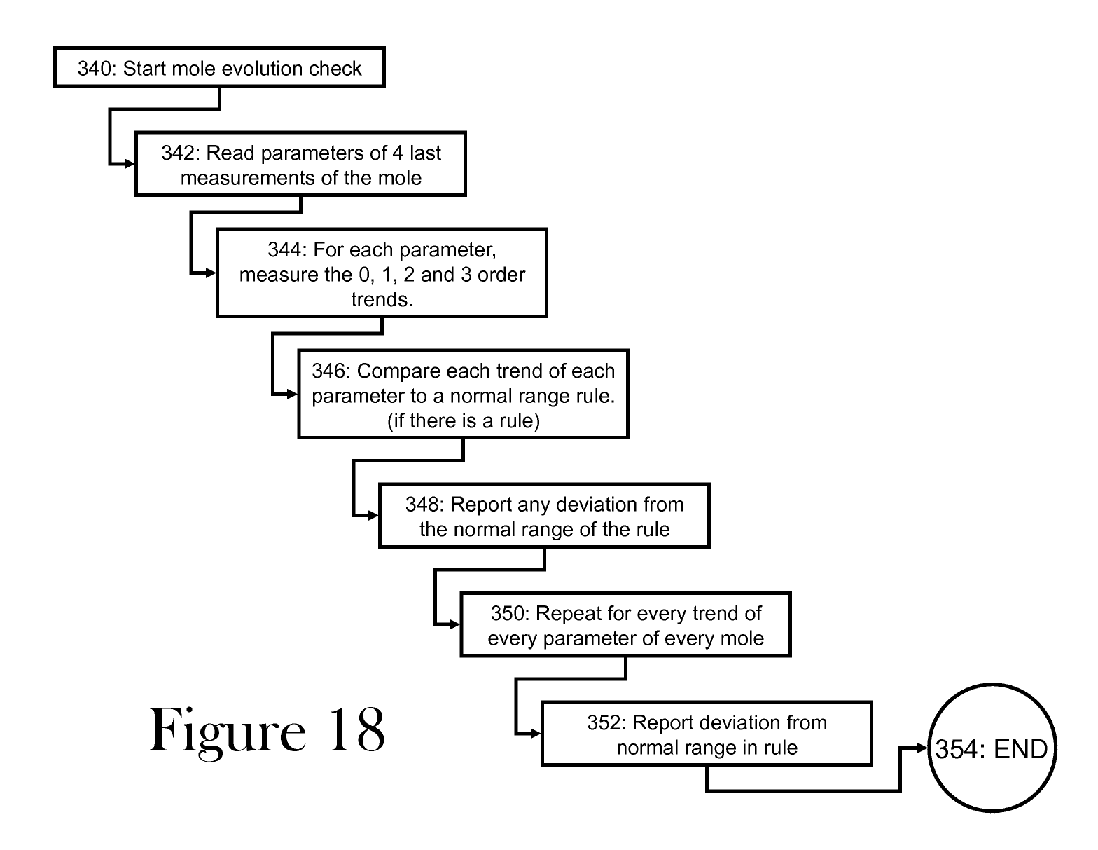

Referring to FIG. 18, that illustrates a flowchart of the process of mole evaluation, according to an embodiment of the invention.

The system reads 342 the parameter vectors of the (typically 4) last measurements of a given mole.

For each of the parameters, the system calculates 344 the zero order (average value), the 1st order (slope), the 2nd order (first derivative), the 3rd order (second derivative).

The system then compares 346 each of the trends in each of the parameters to a predefined normal-range rule. Two examples of rules are “the diameter of a mole changes by less than 4% per month” and “The difference between the color of the band (see FIG. 13) and the color of the periphery is less than 4% in both hue and saturation”.

Any deviation from any of the rules is reported 348. Repeat 350, 352 the evaluation process for each of the trends, in each of the parameters in each of the mole.

The host system that serves the capturing device (a personal computer or an embedded processor in the capturing device) reacts to such alerts according to some policy—for example: issues an email alert to a caregiver: “Mole no. 3, border irregularity increased by 12% in the last 60 days”.

FIG. 19A illustrate an alternative method for determining the topography of the mole according to an embodiment of the invention, that is different than the active stereo method described hereinabove. In this method, two light sources 360 and 362 (such as a light emitting diode—LED) are used to illuminate the mole within the field of view. For all the parameters except for the topography, both light sources are turned on, ensuring good an even illumination of the mole. For topography measurement, the two LED's are turned on alternatively, and the image of the mole is captured twice. The shading of parts of the mole will depend on the orientation of the surface related to the line of illumination. When LED 362 is on and LED 360 is off, areas 376 and 372 will be better illumined than areas 370 and 374. (Areas 368 and 378 are external to the mole contour and are not considered).

When the LED's are switched (in FIG. 19B), and LED 364 is on and LED 366 is off, area 380 becomes more illuminated and 382 becomes darker.

The analysis of the two shaded area provides information about the topography of the mole. This information can be used for determination of peaks and slopes.

Some of the embodiments of the invention, as disclosed above, require a dedicated camera and requires on-line connection to a computer. It is however noted that many users cannot be connected to a computer at all times, and would not want to carry an extra device on them when they are outdoors.

Other users may have a web camera connected to their computer, and would prefer to use that camera and not purchase a dedicated camera for this application,

It would also be desirable to offer a user who owns a web camera that can focus on close targets, an opportunity to use that camera for the utilizations disclosed above.

Therefore, some embodiments of the invention are now presented, that teach systems and methods for implementing the full functionality of the previously discussed embodiments without the need to carry a special camera and without the need to connect the device to a personal computer for processing and communication.

In one embodiment of the present invention, a pocket computer that contains a processor, a mass storage device and a close-up camera—such as the HTC Kjam pocket PC, Nokia N73,N95 camera phone contains a software application the performs essentially all the functions of the camera disclosed above. The images are analyzed by one or more applications running on the pocket PC and the history of the moles is saved in the local storage of the mobile phone or on an attached removable device such as an SD storage card.

A calibration fixture, including some of the elements disclosed in relation to the above disclosed systems, is placed according to an embodiment of this invention, between the skin of the user and the face of the mobile phone. The fixture determines a fixed distance between the camera and the skin provides color calibration and provides light source for angular illumination of the mole.

The operation of the system is identical to the operation of systems described above, where the local processor of the mobile phone performs the functions of the host as described in relation to FIG. 7.

This enables both mobility and availability of the sensing device, that is typically available to the user at all times, and enables him or her to do the testing practically everywhere.

In another embodiment of the present invention, the support fixture is used with an on-line web camera, so that there is no need for a dedicated camera, and the processing is done in a personal computer.

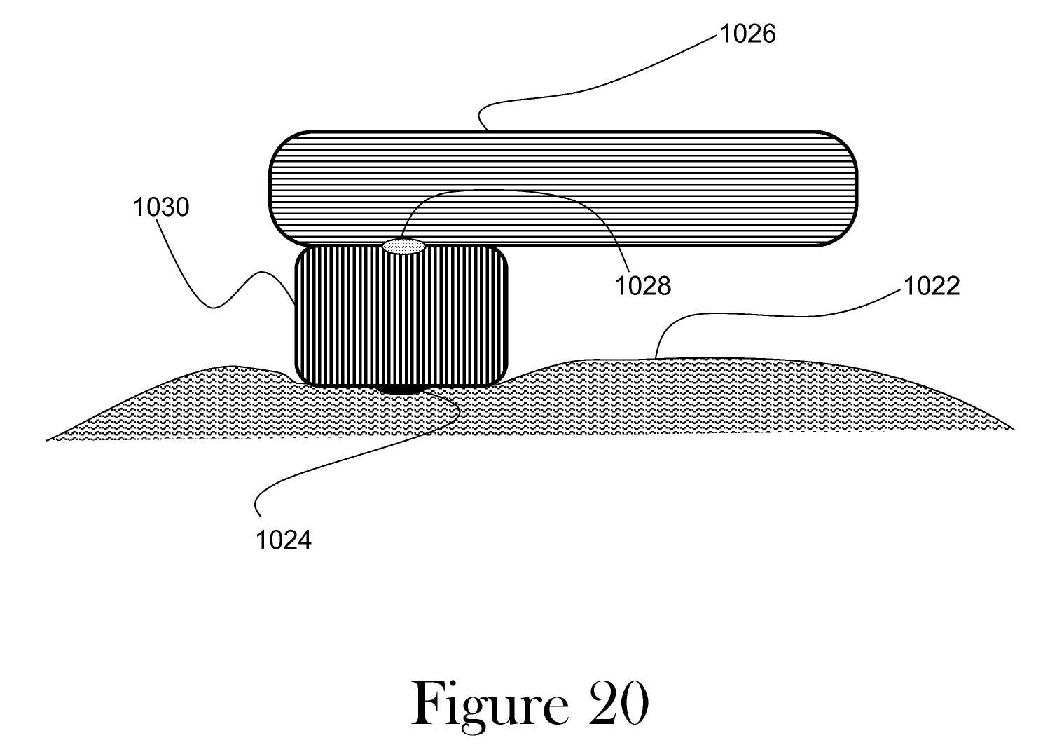

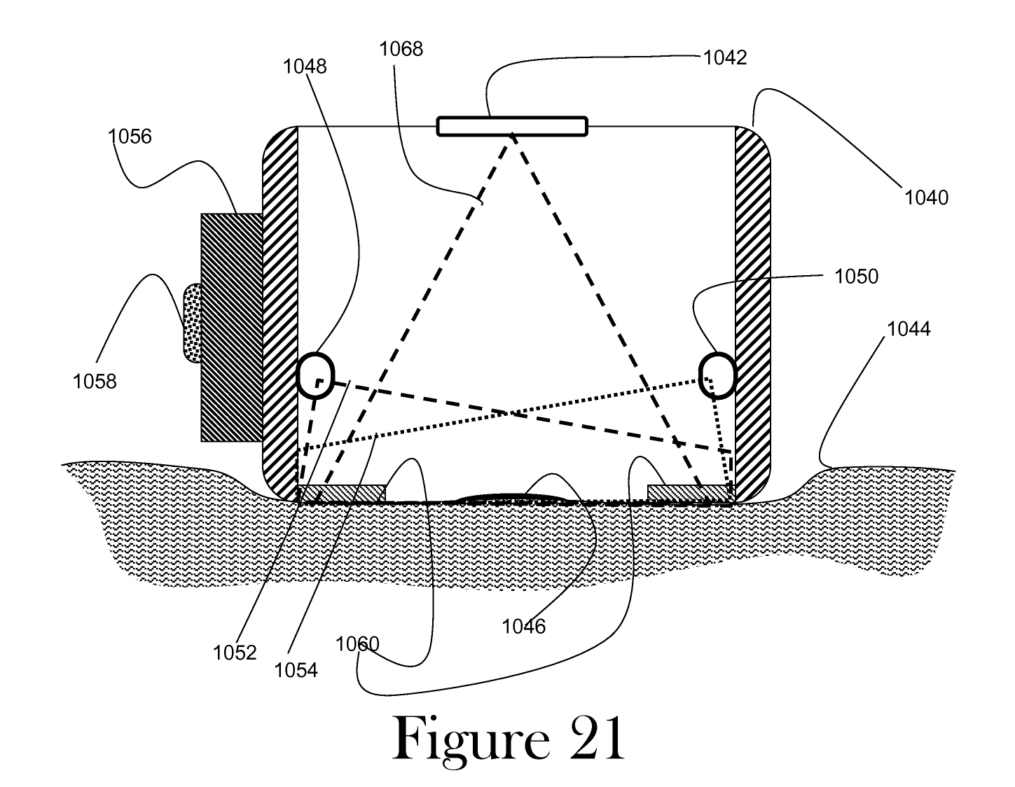

FIG. 20 illustrate layout of the components of the system, according to an embodiment of the invention. A mobile phone 1026 equipped with a camera 1028 is placed on top of a calibration fixture 1030 that is in turn placed over a mole 1024 on the skin 1022 of a user. The calibration fixture is disclosed in relation to FIG. 21. The user typically uses one hand to hold place the fixture on the skin and hold it, and another hand to hold the mobile phone on the fixture and operate it.

FIG. 21 illustrate a vertical cross section through a calibration fixture 1040 that has the general shape of a short and wide tube.

The phone is positioned above the fixture so that its camera 1042 is at the center of the top of the cylinder. The fixture is positioned on the skin 1044 so that the mole 1046 is at the center of the bottom of the cylinder. A ring carrying a color calibration pattern, such as shown in reference number 240 of FIG. 13, is attached to the periphery of the bottom of the cylinder and is covered by the camera field of view 1068.

One or more light sources 1048 and 1050 such as a light emitting diode is installed on the wall of the cylinder, our of the field of view of the camera, and is illuminating the bottom of the cylinder and the mole. By acting separately, the light sources enable the shadow-topography analysis as described in relation to FIG. 19A and 19B. One illuminator sheds light from one direction 1052 and the other illuminator sheds light from the other direction 1054, creating different shadows that can be used for rough estimation of the topography. When the illuminators work simultaneously they provide even illumination needed for the rest of the analysis.

The power for the illumination can be provided by the phone itself, using a cable (not shown) or by built in batteries in a compartment 1056. Power is turned on and off using a switch 1058.

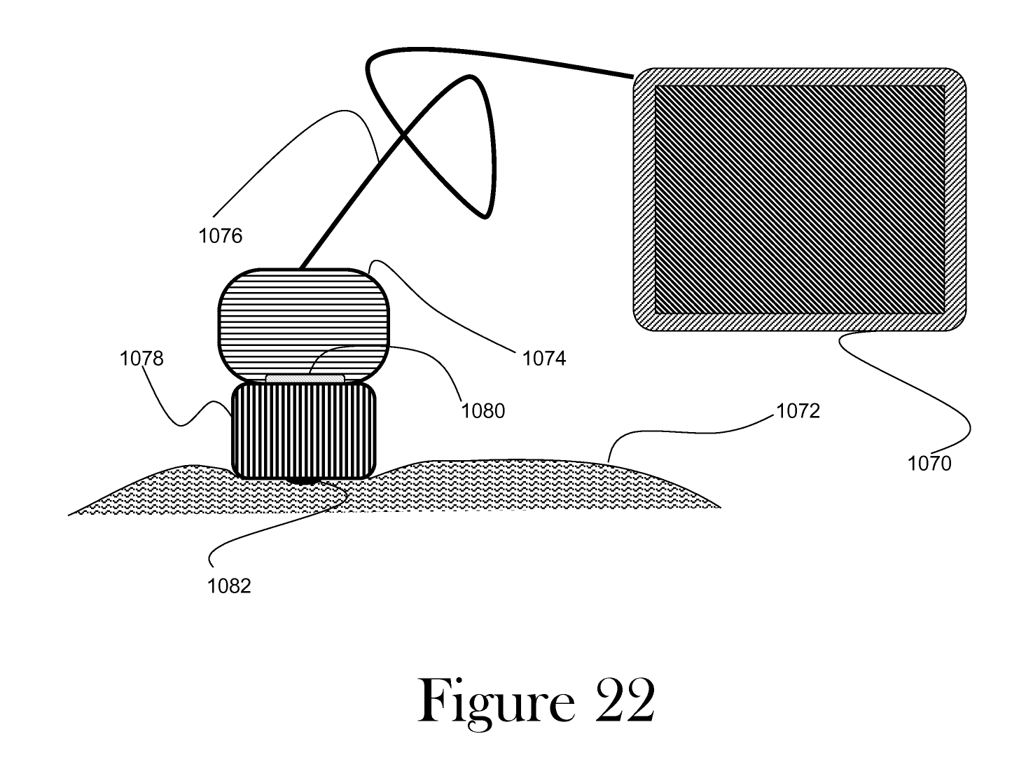

FIG. 22 illustrate a system that utilizes a computer camera, according to an embodiment of the invention.

A personal computer 1070 is connected to a web camera 1074 with a cable 1076. The camera optics 1080 is focused to a short distance. A support fixture 1078, such as described in relation to FIG. 21, is supporting the camera above the mole 1082 on the skin 1072 of the user. The image is transferred directly to the computer and is processed there. Power to the support fixture is provided from the computer via, typically, a USB cable.

According to an embodiment of the invention, a portable system for monitoring evolution of at least two moles on a user's body, is disclosed. It will be clear to a person who is skilled in the art that features of the portable system may be combined with features of any of the previously disclosed systems. The portable system including: a. a mechanism for automatic detection of a mole in a field of view; b. a mechanism for automatic measurement of at least two independent parameters of a mole by its visual appearance; c. a mechanism for automatic identification of a mole, by its measured parameters, among a database of at least two moles, and d. a mechanism for automatic detection of trends of change in at least one of the measured parameters along a sequence of at least three measurements of a mole.

According to an embodiment of the invention, one of the parameter used by the portable system includes a measure of the three dimensional topography of said mole.

According to an embodiment of the invention, the portable system includes a mechanism for parameterization of said three dimensional topography using active stereo.

According to an embodiment of the invention, the portable system includes a mechanism for parameterization of said three dimensional topography using the shadowing of at least two switching light sources.

According to an embodiment of the invention, the portable system includes a mechanism for parameterization of said three dimensional topography using a stereoscopic imaging from at least two directions.

According to an embodiment of the invention, the portable system includes means for automatic registration of a mole within the field of view.

According to an embodiment of the invention, the portable system includes means for color calibration of the mole against instrumental errors, by comparison to a color palette captured in the field of view.

According to an embodiment of the invention, the portable system includes means for color calibration of the mole against tanning effects, by comparison to the background skin around the mole.

According to an embodiment of the invention, the portable system includes a mechanism to modulate said trends by known risk factors of the user.

It is noted that, according to an embodiment of the invention, those risk factors being at least one selected from a group including: eye color, hair color and family history.

According to an embodiment of the invention, the portable system includes a mechanism to reduce the visual noise caused by hair obscuring said mole.

According to an embodiment of the invention, the visual noise reduction mechanism includes a mechanism for moving the hair between a sequence of snapshots.

According to an embodiment of the invention, the visual noise reduction mechanism includes defocusing of said hair.

According to an embodiment of the invention, a diagnostic system is disclosed. It will be clear to a person who is skilled in the art that features of the diagnostic system may be combined with features of any of the previously disclosed systems. The diagnostic system includes a mechanism for detection of difference in redness between a first area and a second area of the human skin.

According to an embodiment of the invention, the first area is a ring around a mole, and said second area is skin further remote from the mole.

According to an embodiment of the invention, the first area is a one of a pair of symmetric organs, and said second area is the other one of the same pair of symmetric organs.

According to an embodiment of the invention, a mole monitoring system is disclosed. It will be clear to a person who is skilled in the art that features of the mole monitoring system may be combined with features of any of the previously disclosed systems. The mole monitoring system includes: a. a general purpose digital camera, selected from a group including a web camera, a mobile phone camera and a general purpose digital camera; b. a calibration fixture comprising an illuminator; and c. a processor operational to parameterize mole images.

According to an embodiment of the invention, the fixture includes a color calibration pallet

According to an embodiment of the invention, the fixture being instrumental to maintain a fixed distance between said camera and said mole.

According to an embodiment of the invention, the illuminator is powered by a mobile phone.

According to an embodiment of the invention, the illuminator is powered by a cable connected to a computer.

According to an embodiment of the invention, illuminator includes a battery.

According to an embodiment of the invention, the camera is embedded in a mobile phone that includes a computing system. It is noted that, according to an embodiment of the invention, the computing system includes mass storage. According to an embodiment of the invention, the computing system is operative to analyze mole images and extract parameters from them. According to an embodiment of the invention, the said computing system is operative to store a history of said mole parameters. According to an embodiment of the invention, the computing system is operative to detect parameters that requires reporting According to an embodiment of the invention, the computing system is operative to automatically generate report messages related to the state of said parameters

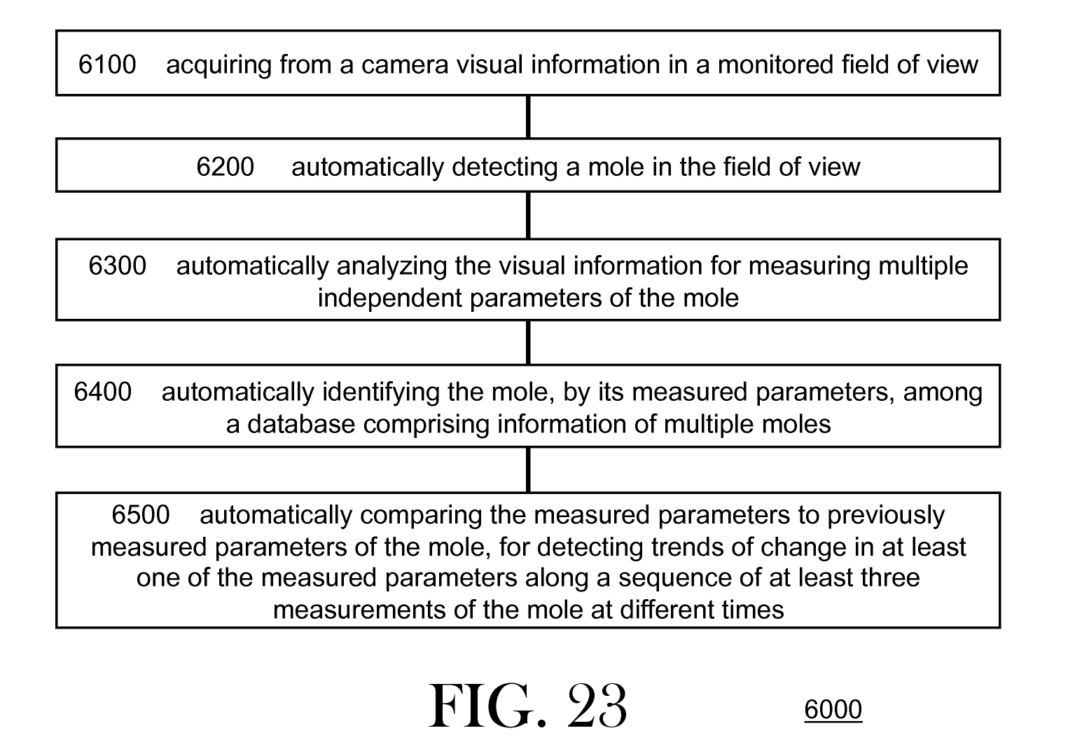

FIG. 23 illustrates a process 6000 that may be implemented by a computer program product comprising a non-transient computer readable medium that includes instructions for the implementation of the process, according to an embodiment of the invention. The process includes:

i. Stage 6100 of acquiring from a camera visual information in a monitored field of view;

ii. Stage 6200 of automatically detecting a mole in the field of view;

iii. Stage 6300 of automatically analyzing the visual information for measuring multiple independent parameters of the mole;

iv. Stage 6400 of automatically identifying the mole, by its measured parameters, among a database including information of multiple moles; and

v. Stage 6500 of automatically comparing the measured parameters to previously measured parameters of the mole, for detecting trends of change in at least one of the measured parameters along a sequence of at least three measurements of the mole at different times.

It is noted that the computer program product may be implemented in the relevant aforementioned systems. The various stages may be implemented by a processor of any of the relevant aforementioned systems, and may also implemented by a group of processors—either of a single unit (e.g. a PC) or of multiple units (e.g. some of the stages implemented by a processor of a mobile phone, while some of the stages implemented by a processor of a personal computer or of a remote computer that receives information from the mobile phone processor over wireless communication channel).

According to an embodiment of the invention, the instructions for analyzing includes instructions for analyzing the visual information for measuring a three dimensional topography of the mole. It is noted that in various embodiments of the invention, other parameters discussed above may also be determined in the analysis (e.g. border features, color, size, shape, symmetry, etc.).

According to an embodiment of the invention, the non-transient computer readable medium further stores instructions for parameterizing the three dimensional topography using at least one method out of a group of techniques consisting of: active stereo, shadowing of at least two switching light sources and stereoscopic imaging.

According to an embodiment of the invention, the non-transient computer readable medium further stores instructions for automatically registering a mole within the field of view.

According to an embodiment of the invention, the non-transient computer readable medium further stores instructions for calibrating a color of the mole by comparing the mole visual information to visual information of a known color reference palette that is also captured in the field of view.

According to an embodiment of the invention, the non-transient computer readable medium further stores instructions for detecting trends are responsive to known risk factors of the user that include one or more risk factor selected from a group consisting of eye color, hair color and family history. For example, bright eye color indication may lead to higher risk factor estimation, and thus to increased attention to trends and deviations during the assessment of trends.

According to an embodiment of the invention, the non-transient computer readable medium further stores instructions for reducing visual noise in the acquired visual information that is caused by hair obscuring said mole by:

i. receiving visual information of images acquired by the camera at different times in which air blown by an air blower moved the hair between different locations; wherein the hair covers different segments of the field of view while being positioned at different locations;

ii. extracting information of pixels from the multiple images based on an amount of hair information included in the pixels; and

iii. generating a synthetic image of the field of view, in which different pixels are extracted from different images of the multiple images.

According to an embodiment of the invention, the non-transient computer readable medium further stores instructions for detecting differences in redness between multiple imaged areas.

According to an embodiment of the invention, the instructions for detecting differences in redness include instructions for detecting differences in redness between a first area that is a ring shaped area around the mole, and a second area that is further remote from the mole.

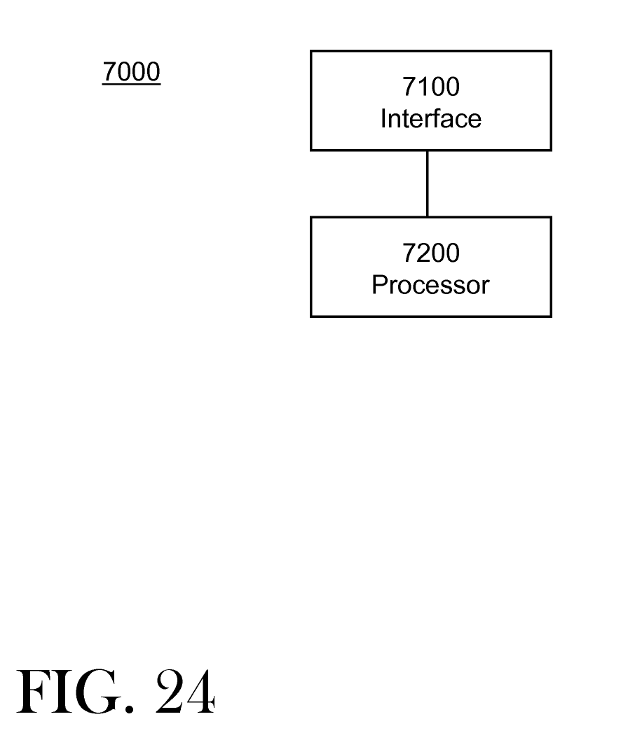

FIG. 24 illustrates portable mole evolution monitoring system 7000, according to an embodiment of the invention. System 7000, that is operational for mole evolution monitoring, includes:

i. Interface 7100 for acquiring from a camera (e.g. camera 2200, camera 5200, or other of the aforementioned cameras) visual information in a monitored field of view;

ii. Processor 7200, configured to: automatically detect a mole in the field of view; automatically analyze the visual information for measuring multiple independent parameters of the mole; automatically identify the mole, by its measured parameters, among a database including information of multiple moles; automatically compare the measured parameters to previously measured parameters of the mole, for detecting trends of change in at least one of the measured parameters along a sequence of at least three measurements of the mole at different times.

It is noted that various embodiments of system 7000 may implement carious embodiments of process 6000, even if not explicitly elaborated.

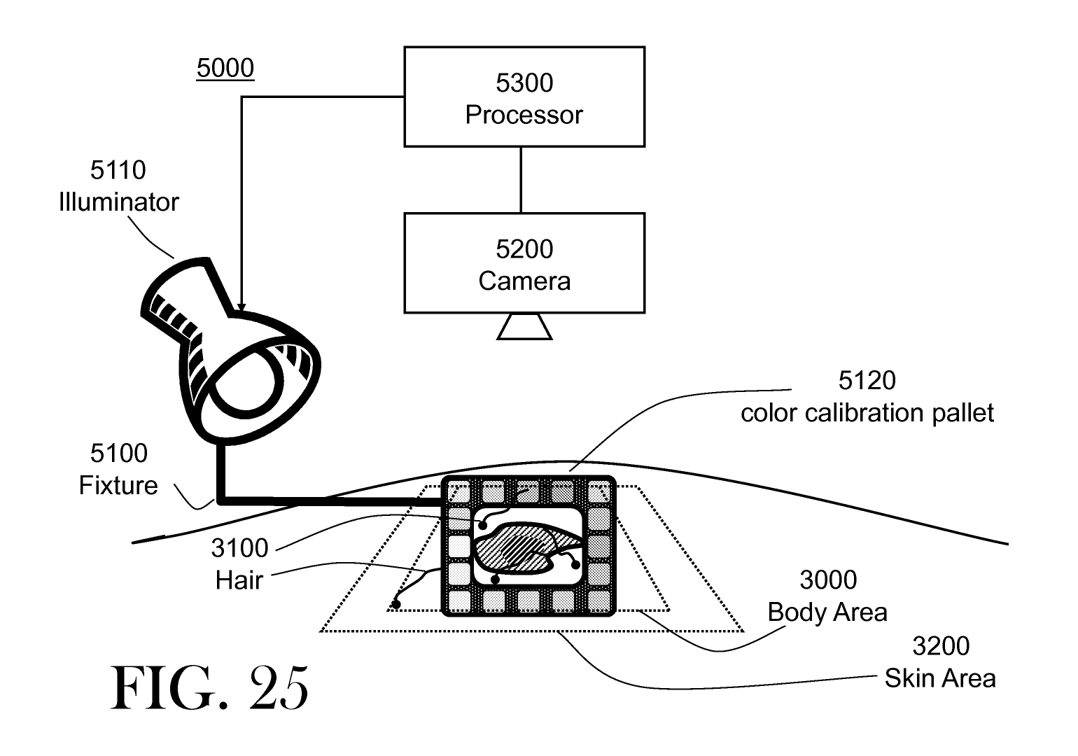

FIG. 25 illustrates mole monitoring system 5000, according to an embodiment of the invention. Mole monitoring system 500 includes:

i. calibration fixture 5100 including an illuminator 5110 that is operational to illuminate skin area 3200 that includes a mole;

ii. digital camera 5200 operational to acquire at least one image of body area 3000 that is included in the illuminated skin area 3200 and which includes the mole and its environment; and

iii. processor 5300 operational to analyze the at least one image of the mole and to determine values for parameters of the mole.

It is noted that, according to an embodiment of the invention, mole monitoring system 5000 may be portable, e.g. incorporated into a cellular phone—wherein the illumination may be an embedded illumination or an external illumination, maybe partly portable (e.g. combination of a cellular phone and a PC), a dedicated system, and so forth.

According to an embodiment of the invention, camera 5200 may be, by way of example, a web camera (e.g. of a desktop computer, integrated into a lap-top computer, and so forth), a mobile phone camera, a general purpose digital camera, and so forth.

Camera 5200 may and may not be controlled by processor 5300. Illuminator 5110 may and may not be controlled by processor 5300

It is noted that, according to various embodiment of the invention, processor 5300 may also function as processor 2300 of the above disclosed system 2000, and that system 2000 and system 5000 may be integrated into a single system.

According to an embodiment of the invention, fixture 5100 further includes (or supports) color calibration pallet 5120, wherein digital camera 5200 is further operational to acquire the at least one image that includes visual information of color calibration pallet 5120 (and of skin area 3000, which may be partly covered by color calibration pallet 5120), wherein processor 5300 is further operational to calibrate colors of the at least one image in response to visual information of the color calibration pallet 5120 included in the at least one image.

According to an embodiment of the invention, fixture 5100 is further instrumental to maintain a fixed distance between digital camera 5200 and the mole.

According to an embodiment of the invention, digital camera 5200 is embedded in a mobile phone that further includes a computing system. According to various embodiments of the invention, the mobile phone (or other, possibly dedicated, portable system) may further include some or all of the following components: processor 5300, air blower 2100, illuminator 5100, etc. It is noted that, according to an embodiment of the invention, color calibration pallet 5120 may not be fixed to the fixture by a stand alone unit, that a user can locate in the vicinity of the mole before imaging.

According to an embodiment of the invention, the computing system of the mobile phone includes mass storage for storing image data of the at least one image.

According to an embodiment of the invention, the computing system of the mobile phone is operative to analyze mole images and extract parameters from them.

According to an embodiment of the invention, the computing system of the mobile phone is further operative to store a history of the mole parameters.

According to an embodiment of the invention, the computing system of the mobile phone is further operative to detect deviation in one or more of the mole parameters

According to an embodiment of the invention, the computing system of the mobile phone is further operative to automatically generate report messages pertaining to the state of the mole parameters.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An imaging system for imaging a body area, the system comprising:
an air blower for moving hair of the body area between different locations by blowing air toward the hair; wherein the hair covers different segments of the body area while being positioned at different locations;
a camera, configured to acquire multiple images of the body area while the hair is positioned in different locations due to the blowing of air; and
a processor, configured to:
receive the multiple images;
extract information of pixels from the multiple images based on an amount of hair information included in the pixels; and
generate a synthetic image of the body area, in which different pixels are extracted from different images of the multiple images.

2. The imaging system of claim 1, wherein the processor is further configured to select pixels according to the brightness of the corresponding pixels of the multiple images.

3. A computer program product comprising a non-transient computer readable medium that stores instructions for:
acquiring from a camera visual information in a monitored field of view;
automatically detecting a mole in the field of view;
automatically analyzing the visual information for measuring multiple independent parameters of the mole;
automatically identifying the mole, by its measured parameters, among a database comprising information of multiple moles; and
automatically comparing the measured parameters to previously measured parameters of the mole, for detecting trends of change in at least one of the measured parameters along a sequence of at least three measurements of the mole at different times.

4. The computer program product of claim 3, wherein the instructions for analyzing comprises instructions for analyzing the visual information for measuring a three dimensional topography of the mole.

5. The computer program product of claim 4, wherein the non-transient computer readable medium further stores instructions for parameterizing the three dimensional topography using at least one method out of a group of techniques consisting of: active stereo, shadowing of at least two switching light sources and stereoscopic imaging.

6. The computer program product of claim 3, the non-transient computer readable medium further stores instructions for automatically registering a mole within the field of view.

7. The computer program product of claim 3, wherein the non-transient computer readable medium further stores instructions for calibrating a color of the mole by comparing the mole visual information to visual information of a known color reference palette that is also captured in the field of view.

8. The computer program product of claim 3, wherein the non-transient computer readable medium further stores instructions for detecting trends are responsive to known risk factors of the user that comprise one or more risk factor selected from a group consisting of eye color, hair color and family history.

9. The computer program product of claim 3, wherein the non-transient computer readable medium further stores instructions for reducing visual noise in the acquired visual information that is caused by hair obscuring said mole by:
receiving visual information of images acquired by the camera at different times in which air blown by an air blower moved the hair between different locations; wherein the hair covers different segments of the field of view while being positioned at different locations;
extracting information of pixels from the multiple images based on an amount of hair information included in the pixels; and
generating a synthetic image of the field of view, in which different pixels are extracted from different images of the multiple images.

10. The computer program product of claim 3, wherein the non-transient computer readable medium further stores instructions for detecting differences in redness between multiple imaged areas.

11. The computer program product of claim 10, wherein the instructions for detecting differences in redness comprise instructions for detecting differences in redness between a first area that is a ring shaped area around the mole, and a second area that is further remote from the mole.

12. A mole monitoring system comprising:
a calibration fixture comprising an illuminator that is operational to illuminate a skin area that comprises a mole;
a digital camera operational to acquire at least one image of a body area that is comprised in the illuminated skin area and which comprises the mole and its environment; and
a processor operational to analyze the at least one image of the mole and to determine values for parameters of the mole.

13. The system of claim 12, wherein the fixture further comprise a color calibration pallet, wherein the digital camera is further operational to acquire the at least one image that includes visual information of the color calibration pallet, wherein the processor is further operational to calibrate colors of the at least one image in response to visual information of the color calibration pallet comprised in the at least one image.

14. The system of claim 12, wherein the fixture is further instrumental to maintain a fixed distance between the digital camera and the mole.

15. The system of claim 12, wherein the digital camera is embedded in a mobile phone that further comprises a computing system.

16. The system of claim 15, wherein the computing system of the mobile phone comprises mass storage for storing image data of the at least one image.

17. The system of claim 15, wherein the computing system of the mobile phone is operative to analyze mole images and extract parameters from them.

18. The system of claim 17, wherein the computing system of the mobile phone is further operative to store a history of the mole parameters.

19. The system of claim 18, wherein the computing system of the mobile phone is further operative to detect deviation in one or more of the mole parameters

20. The system of claim 19, wherein the computing system of the mobile phone is further operative to automatically generate report messages pertaining to the state of the mole parameters.
